(19) United States
(12) Patent Application Publication
(10) Pub. No.: US 2023/0098438 A1
(43) Pub. Date: Mar. 30, 2023

(54) OXAZINE-BASED WATER-SOLUBLE FLUOROPHORE COMPOUNDS FOR IN VIVO NERVE IMAGING

(71) Applicant: Oregon Health & Science University, Portland, OR (US)

(72) Inventors: Summer L. Gibbs, West Linn, OR (US); Lei G. Wang, Portland, OR (US); Connor W. Barth, Portland, OR (US)

(73) Assignee: Oregon Health & Science University, Portland, OR (US)

(21) Appl. No.: 17/790,698

(22) PCT Filed: Dec. 21, 2020
(86) PCT No.: PCT/US2020/066393

§ 371 Date: Jul. 1, 2022

Related U.S. Application Data
(60) Provisional application No. 62/956,614, filed on Jan. 2, 2020.

(51) Int. Cl. C07D 265/38

(52) U.S. Cl. CPC C07D 265/38

(57) ABSTRACT

This invention provides novel oxazine-based, water soluble fluorophore compounds useful in in vivo nerve imaging, as well as compositions comprising them and methods for their use.

### CROSS-REFERENCE TO RELATED APPLICATIONS

This is the 371 National Phase of PCT/US2020/066393, filed Dec. 21, 2020, which claims priority to and the benefit of the earlier filing of U.S. Provisional Application No. 62/956,614, filed on Jan. 2, 2020, which is incorporated by reference herein in its entirety.

### STATEMENT REGARDING FEDERALLY SPONSORED RESEARCH OR DEVELOPMENT

This invention was made with government support under grant R01 EB021362 awarded by the National Institutes of Health. The government has certain rights in the invention.

### FIELD OF THE DISCLOSURE

This invention concerns novel oxazine-based, water soluble fluorophore compounds useful in in vivo nerve imaging, as well as compositions comprising them and methods for their use.

### BACKGROUND OF THE INVENTION

Over 300 million surgeries are performed worldwide each year. Despite many recent advances in the treatment of cancer and other diseases, surgery remains the most effective treatment option for a number of diseases and injuries. The ultimate goal of surgery is to remove or repair tissues while minimizing comorbidities by preserving vital structures such as nerves and blood vessels. Recent technological advances including minimally invasive robot assisted laparoscopic surgery have improved outcomes and made it possible to perform difficult procedures robustly with minimal risk. Furthermore, preoperative three-dimensional imaging technologies such as magnetic resonance imaging (MRI) and computed tomography (CT) have vastly improved diagnostic accuracy, staging, and preoperative planning.

While advances have been made, identifying vital structures for preservation (e.g., nerves) or tissue for complete resection (e.g., tumors) during surgical procedures remains difficult. Nerve identification and sparing can be difficult intraoperatively due to variations in patient anatomy and often little ability for direct nerve visualization in the surgical field. Currently, intraoperative nerve detection is performed through a combination of naked eye visualization, palpation, and electromyographic monitoring. Several imaging modalities have been utilized in clinical studies for nerve detection including ultrasound, optical coherence tomography, and confocal endomicroscopy. However, these lack specificity, resolution, and wide-field imaging functionality, making it difficult to identify nerve tissues in real time. As a result, nerve damage continues to plague surgical outcomes. latrogenic nerve injury affects up to 63 million patients worldwide annually, causing acute and chronic pain as well as impairment or loss of motor and sensory function. Radical prostatectomy (RP), a surgical procedure involving removal of the entire prostate as a prostate cancer cure, is particularly plagued by nerve damage. Furthermore, while minimally invasive methods, such as robotic assisted RP, can achieve equivalent cancer control to open RP while resulting in decreased blood loss, lower transfusion rate, and faster convalescence, these advances provide no benefit in nerve-sparing outcomes and in fact, remove the ability to directly palpate the tissue.

An imaging modality capable of wide field, real time identification of nerve tissues intraoperatively would greatly benefit surgeons in nerve preservation and reduce rates of iatrogenic nerve injury, improving quality of life for patients post-surgery.

Currently, no NIR nerve-specific fluorophore exists and further fluorophore development is required to obtain a proper candidate for clinical translation. Several classes of nerve specific fluorophores have been studied for FGS. See, for instance: Gibbs-Strauss et al. Molecular imaging 10, 91-101 (2011); Wu et al. Journal of medicinal chemistry 51, 6682-6688 (2008); Wang et al. The Journal of Histochemistry and Cytochemistry: official journal of the Histochemistry Society 58, 611-621 (2010); Gibbs et al. PloS ONE 8, e73493 (2013); Stankoff et al. Proceedings of the National Academy of Sciences of the United States of America 103, 9304-9309 (2006); Cotero et al., Molecular Imaging and Biology: MIB: the official publication of the Academy of Molecular Imaging 14, 708-717 (2012); Cotero et al. PloS ONE 10, e0130276 (2015); Bajaj et al. The Journal of Histochemistry and Cytochemistry: Official Journal of the Histochemistry Society 61, 19-30 (2013); Gibbs-Strauss et al. Molecular imaging 9, 128-140 (2010); Meyers et al. The Journal of Neuroscience: the Official Journal of the Society for Neuroscience 23, 4054-4065 (2003); Wang et al. The Journal of Neuroscience: the official journal of the Society for Neuroscience 31, 2382-2390 (2011); and Park et al. Theranostics 4, 823-833 (2014). Of these, Oxazine 4 is the most promising candidate for development, showing high nerve-specificity and red shifted absorption and emission spectra close to the NIR (Park et al. Theranostics 4, 823-833 (2014)).

Useful oxazine nerve-sparing fluorophores are disclosed in International Application PCT/US2019/045347, but there remains a need for such compounds for use in aqueous compositions.

### SUMMARY OF THE INVENTION

Provided is a compound of Formula (I):

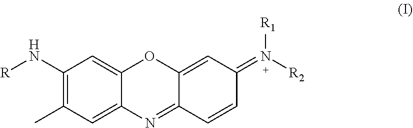

wherein:

R is a straight or branched alkyl chain of from 2 to 12 carbon atoms;

R₁ is selected from the group of methyl, ethyl, n-propyl, isopropyl, —(CH₂)ₙ₁—SO₃⁻, —(CH₂)ₙ₁—N⁺(CH₃)₃, —CH₂—CH₂—O—X₁, —CH₂—CH₂—O—[CH₂—CH₂—O]ₙ₂—X₁, —CH₂—CH₂—CH₂—O—X₁, and —CH₂—CH₂—CH₂—O—[CH₂—CH₂—CH₂—O]ₙ₃—X₁;

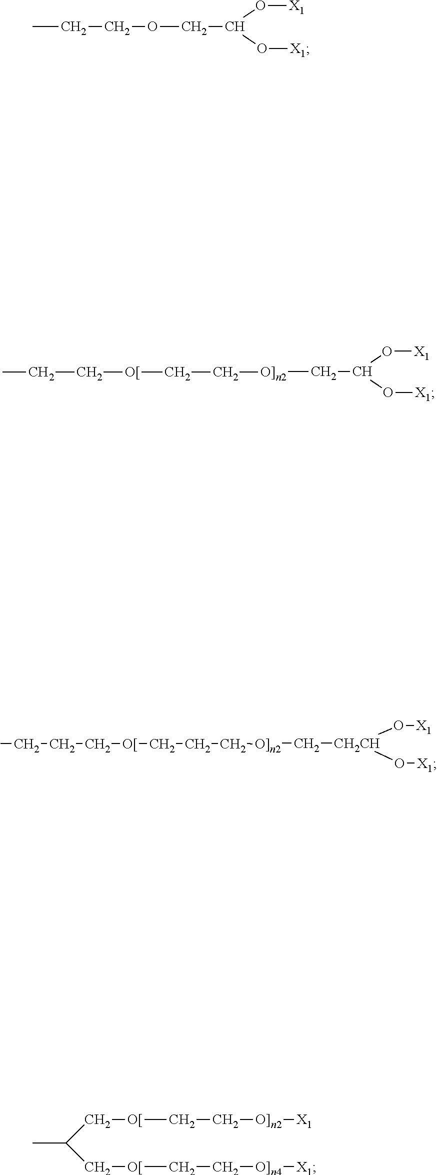

R₂ is selected from the group of —(CH₂)ₙ₁—SO₃⁻, —(CH₂)ₙ₁—N⁺(CH₃)₃, —CH₂—CH₂—O—X₁, —CH₂—CH₂—O—[CH₂—CH₂—O]ₙ₄—X₁, —CH₂—CH₂—CH₂—O—X₁, and —CH₂—CH₂—CH₂—O—[CH₂—CH₂—CH₂—O]ₙ₅—X₁;

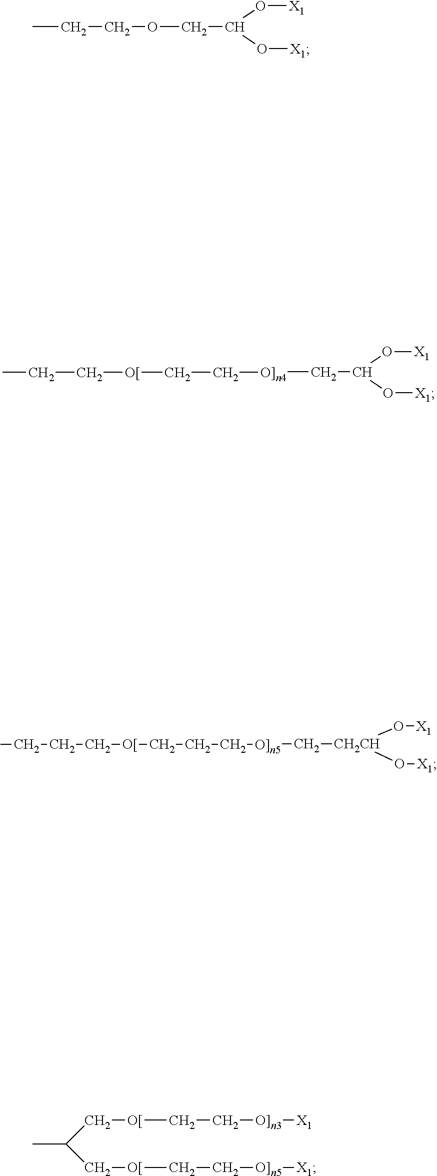

X₁ in each instance is independently selected from C₁-C₆ straight or branched alkyl, C₂-C₆ straight or branched alkenyl, C₁-C₆ straight or branched alkynyl, and —Si(C₁-C₄ alkyl)₃;

n1 is an integer independently selected in each instance from the group of 1, 2, 3, and 4;

n2 is an integer independently selected in each instance from the group of 1, 2, 3, 4, 5, 6, 7, 8, 9, and 10;

n3 is an integer independently selected in each instance from the group of 1, 2, 3, 4, 5, 6, 7, 8, 9, and 10;

n4 is an integer independently selected in each instance from the group of 1, 2, 3, 4, 5, 6, 7, 8, 9, and 10; and n5 is an integer independently selected in each instance from the group of 1, 2, 3, 4, 5, 6, 7, 8, 9, and 10;

with the proviso that the sum of n2+n4 is not greater than 10;

with the proviso that the sum of n3+n5 is not greater than 10;

with the proviso that the sum of n2+n5 is not greater than 10; and

with the proviso that the sum of n3+n4 is not greater than 10.

### BRIEF DESCRIPTION OF THE DRAWINGS

FIG. 1 provides comparative fluorescence images of compounds herein administered to tissue.

FIG. 2 provides tabulated spectral and physicochemical properties of screening candidates.

FIG. 3A provides representative photographs, and fluorescence images of the NIR oxazine derivatives after direct application (125 μM in PBS) to exposed sciatic nerves. All images are representative of data collected for n=6 nerve sites per fluorophore.

FIG. 3B depicts the average nerve (white), muscle (black) and adipose (gray) tissue intensities per second were quantified in comparison to an unstained control group.

FIG. 3C depicts quantified nerve SBRs were calculated for comparison between the screening candidates and unstained control group. All quantified data is presented as the mean±standard deviation.

FIG. 4A provides representative photographs and fluorescence images of the NIR water-soluble nerve-specific candidate LGW13-98 after systemic administration at various dosages.

FIG. 4B represents the comparison of quantified nerve, muscle and adipose tissue intensities per second.

FIG. 4C represents the comparison of quantified nerve, muscle and adipose tissue intensities calculated nerve-to-muscle (middle) and

FIG. 4D represents the comparison of quantified nerve, muscle and adipose tissue nerve-to-adipose ratios.

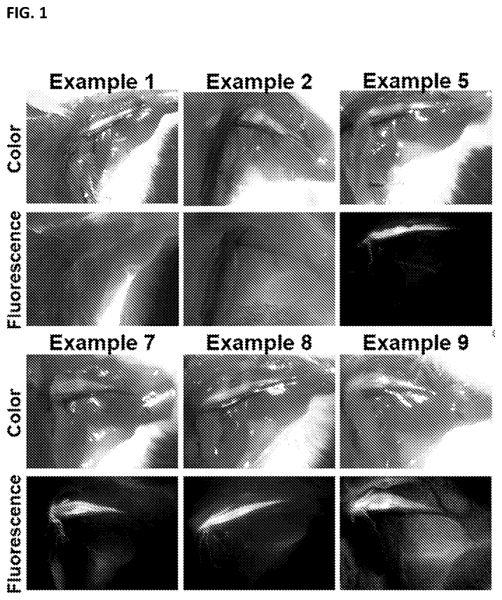

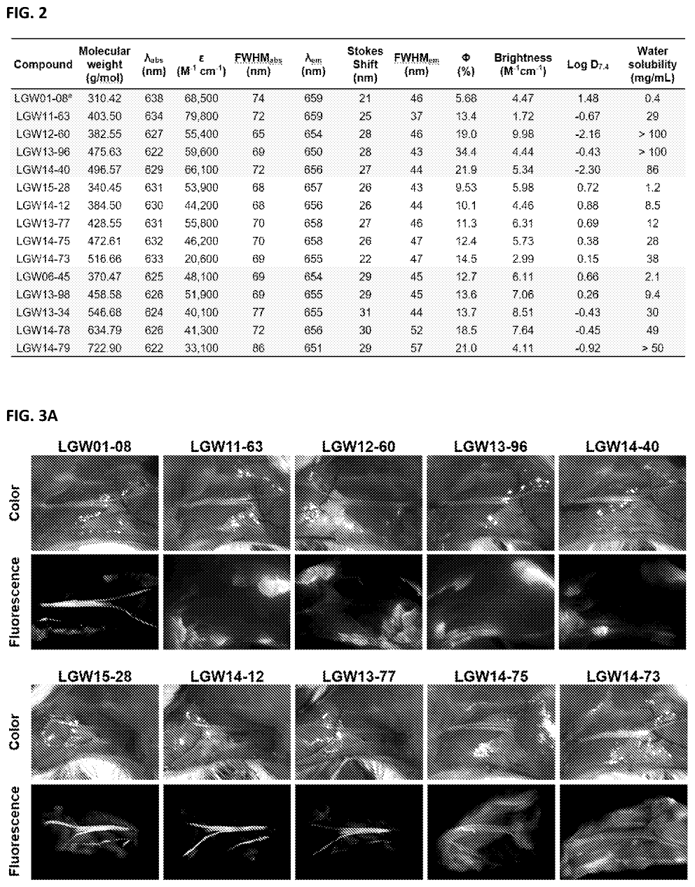

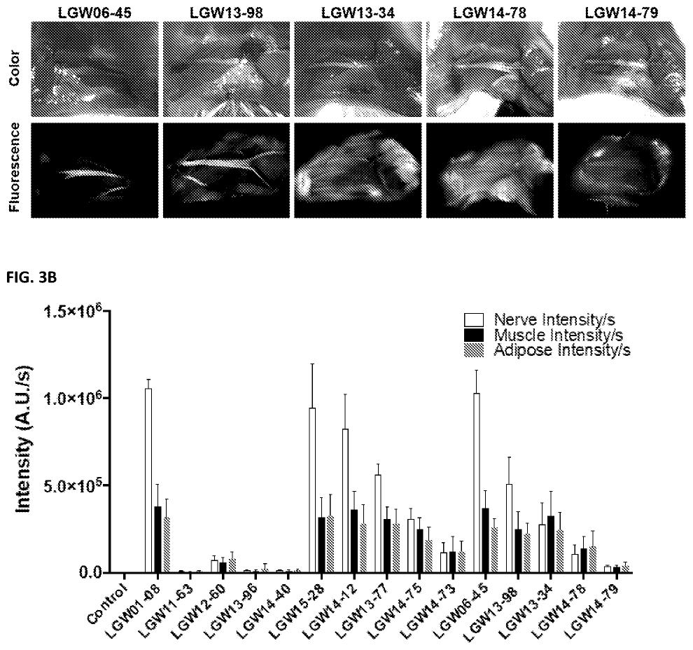

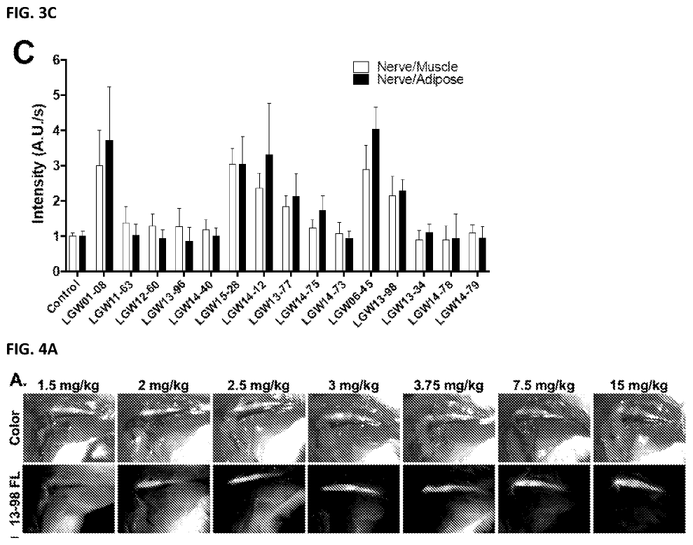

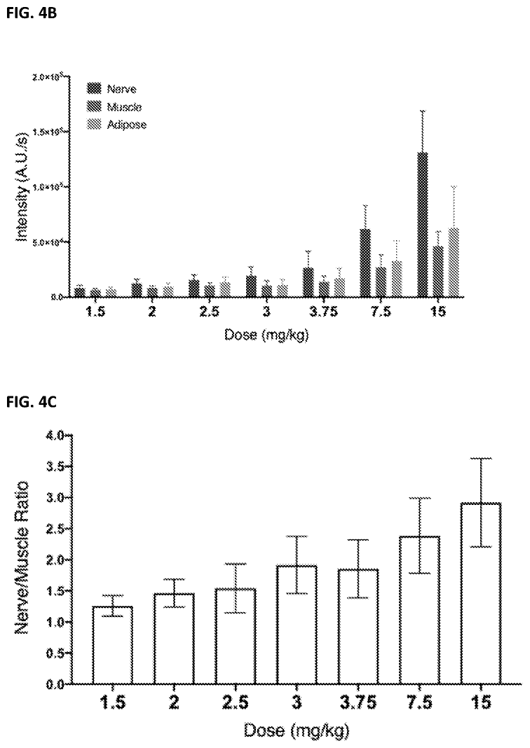

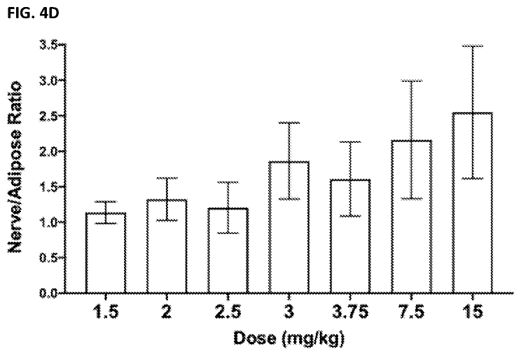

### DETAILED DESCRIPTION OF THE INVENTION

A number of embodiments are provided for a compound of Formula (I).

Also provided is a compound of Formula (I), wherein R is a straight or branched alkyl chain of from 2 to 10 carbon atoms in length and R₁, R₂, X₁, n1, n2, n3, n4, and n5 are as defined above.

Also provided is a compound of Formula (I), wherein R is a straight or branched alkyl chain of from 2 to 10 carbon atoms in length; R₁ is selected from methyl, ethyl, n-propyl, and isopropyl; and R₂, X₁, n1, n2, n3, n4, and n5 are as defined above.

Also provided is a compound of Formula (I), wherein R is a straight alkyl chain of from 2 to 10 carbon atoms in length; R₁ is selected from methyl, ethyl, n-propyl, and isopropyl; and R₂, X₁, n1, n2, n3, n4, and n5 are as defined above.

Also provided is a compound of Formula (I), wherein R is a straight or branched alkyl chain of from 2 to 10 carbon atoms in length; R₁ is ethyl; and R₂, X₁, n1, n2, n3, n4, and n5 are as defined above.

Also provided is a compound of Formula (I), wherein R is a straight alkyl chain of from 2 to 10 carbon atoms in length; R₁ is ethyl; and R₂, X₁, n1, n2, n3, n4, and n5 are as defined above.

Also provided is a compound of Formula (I), wherein R is a straight or branched alkyl chain of from 2 to 8 carbon atoms in length and R₁, R₂, X₁, n1, n2, n3, n4, and n5 are as defined above.

Also provided is a compound of Formula (I), wherein R is a straight or branched alkyl chain of from 2 to 8 carbon atoms in length; R₁ is selected from methyl, ethyl, n-propyl, and isopropyl; and R₂, X₁, n1, n2, n3, n4, and n5 are as defined above.

Also provided is a compound of Formula (I), wherein R is a straight alkyl chain of from 2 to 8 carbon atoms in length; R₁ is selected from methyl, ethyl, n-propyl, and isopropyl; and R₂, X₁, n1, n2, n3, n4, and n5 are as defined above.

Also provided is a compound of Formula (I), wherein R is a straight or branched alkyl chain of from 2 to 8 carbon atoms in length; R₁ is ethyl; and R₂, X₁, n1, n2, n3, n4, and n5 are as defined above.

Also provided is a compound of Formula (I), wherein R is a straight alkyl chain of from 2 to 8 carbon atoms in length; R₁ is ethyl; and R₂, X₁, n1, n2, n3, n4, and n5 are as defined above.

Also provided is a compound of Formula (I), wherein R is a straight or branched alkyl chain of from 2 to 6 carbon atoms in length and R₁, R₂, X₁, n1, n2, n3, n4, and n5 are as defined above.

Also provided is a compound of Formula (I), wherein R is a straight or branched alkyl chain of from 2 to 6 carbon atoms in length; R₁ is selected from methyl, ethyl, n-propyl, and isopropyl; and R₂, X₁, n1, n2, n3, n4, and n5 are as defined above.

Also provided is a compound of Formula (I), wherein R is a straight alkyl chain of from 2 to 6 carbon atoms in length; R₁ is selected from methyl, ethyl, n-propyl, and isopropyl; and R₂, X₁, n1, n2, n3, n4, and n5 are as defined above.

Also provided is a compound of Formula (I), wherein R is a straight or branched alkyl chain of from 2 to 6 carbon atoms in length; R₁ is ethyl; and R₂, X₁, n1, n2, n3, n4, and n5 are as defined above.

Also provided is a compound of Formula (I), wherein R is a straight alkyl chain of from 2 to 6 carbon atoms in length; R₁ is ethyl; and R₂, X₁, n1, n2, n3, n4, and n5 are as defined above.

Also provided is a compound of Formula (I), wherein R is a straight or branched alkyl chain of from 2 to 4 carbon atoms in length and R₁, R₂, X₁, n1, n2, n3, n4, and n5 are as defined above.

Also provided is a compound of Formula (I), wherein R is a straight or branched alkyl chain of from 2 to 4 carbon atoms in length; R₁ is selected from methyl, ethyl, n-propyl, and isopropyl; and R₂, X₁, n1, n2, n3, n4, and n5 are as defined above.

Also provided is a compound of Formula (I), wherein R is a straight alkyl chain of from 2 to 4 carbon atoms in length; R₁ is selected from methyl, ethyl, n-propyl, and isopropyl; and R₂, X₁, n1, n2, n3, n4, and n5 are as defined above.

Also provided is a compound of Formula (I), wherein R is a straight or branched alkyl chain of from 2 to 4 carbon atoms in length; R₁ is ethyl; and R₂, X₁, n1, n2, n3, n4, and n5 are as defined above.

Also provided is a compound of Formula (I), wherein R is a straight alkyl chain of from 2 to 4 carbon atoms in length; R₁ is ethyl; and R₂, X₁, n1, n2, n3, n4, and n5 are as defined above.

Within each of the separate embodiments herein concerning Formula (I), there is a further additional embodiment wherein all variables and provisos are as defined for the separate embodiment in question, with the additional proviso that, when R₂ is the moiety:

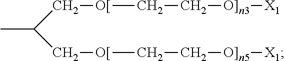

then R₁ is selected from the group of methyl, ethyl, n-propyl, and isopropyl.

Within each of the separate embodiments herein concerning Formula (I), there is also a further additional embodiment wherein all variables and provisos are as defined for the separate embodiment in question, with the additional proviso that, when R₂ is the moiety:

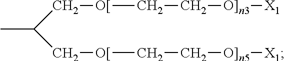

then R₁ is selected from the group of methyl, ethyl, and n-propyl.

Within each of the separate embodiments herein concerning Formula (I), there is also a further additional embodiment wherein all variables and provisos are as defined for the separate embodiment in question, with the additional proviso that, when R₂ is the moiety:

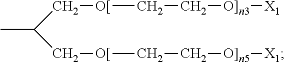

then R₁ is ethyl.

Another embodiment provides a compound of Formula (II):

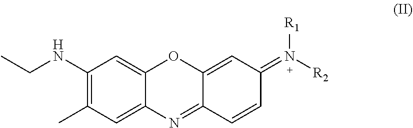

wherein:

R₁ is selected from the group of methyl, ethyl, n-propyl, isopropyl, —(CH₂)ₙ₁—SO₃⁻, —(CH₂)ₙ₁—N⁺(CH₃)₃;

—CH₂—CH₂—O—X₁;

—CH₂—CH₂—O—[CH₂—CH₂—O]ₙ₂—X₁;

—CH₂—CH₂—CH₂—O—X₁; and

—CH₂—CH₂—CH₂—O—[CH₂—CH₂—CH₂—O]ₙ₃—X₁;
R₂ is selected from the group of —(CH₂)ₙ₁—SO₃−, —(CH₂)ₙ₁—N⁺(CH₃)₃;

—CH₂—CH₂—O—X₁;

—CH₂—CH₂—O—[CH₂—CH₂—O]ₙ₄—X₁;

—CH₂—CH₂—CH₂—O—X₁; and

—CH₂—CH₂—CH₂—O—[CH₂—CH₂—CH₂—O]ₙ₅—X₁;

X₁ in each instance is independently selected from C₁-C₆ straight or branched alkyl, C₂-C₆ straight or branched alkenyl, C₁-C₆ straight or branched alkynyl, and —Si(C₁-C₄ alkyl)₃;

n1 is an integer independently selected in each instance from the group of 1, 2, 3, and 4;

n2 is an integer independently selected in each instance from the group of 1, 2, 3, 4, 5, 6, 7, 8, 9, and 10;

n3 is an integer independently selected in each instance from the group of 1, 2, 3, 4, 5, 6, 7, 8, 9, and 10;

n4 is an integer independently selected in each instance from the group of 1, 2, 3, 4, 5, 6, 7, 8, 9, and 10; and n5 is an integer independently selected in each instance from the group of 1, 2, 3, 4, 5, 6, 7, 8, 9, and 10;

with the proviso that the sum of n2+n4 is not greater than 10;

with the proviso that the sum of n3+n5 is not greater than 10;

with the proviso that the sum of n2+n5 is not greater than 10; and

with the proviso that the sum of n3+n4 is not greater than 10.

A further embodiment provides a compound of Formula (II):

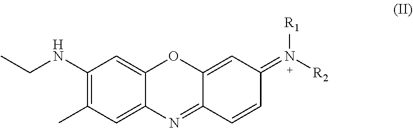

wherein:

R₁ is selected from the group of methyl, ethyl, n-propyl, isopropyl, —(CH₂)ₙ₁—SO₃⁻, —(CH₂)ₙ₁—N⁺(CH₃)₃;

—CH₂—CH₂—O—CH₃;

—CH₂—CH₂—O—CH₂—CH₃,

—CH₂—CH₂—O—[CH₂—CH₂—O]ₙ₂—CH₃;

—CH₂—CH₂—O—[CH₂—CH₂—O]ₙ₂—CH₂—CH₃;

—CH₂—CH₂—CH₂—O—CH₃;

—CH₂—CH₂—CH₂—O—CH₂—CH₃;

—CH₂—CH₂—CH₂—O—[CH₂—CH₂—CH₂—O]ₙ₃—CH₃; and

—CH₂—CH₂—CH₂—O—[CH₂—CH₂—CH₂—O]ₙ₃—CH₂—CH₃;
R₂ is selected from the group of —(CH₂)ₙ₁—SO₃⁻, —(CH₂)ₙ₁—N⁺(CH₃)₃;

—CH₂—CH₂—O—CH₃;

—CH₂—CH₂—O—CH₂—CH₃,

—CH₂—CH₂—O—[CH₂—CH₂—O]ₙ₄—CH₃;

—CH₂—CH₂—O—[CH₂—CH₂—O]ₙ₄—CH₂—CH₃;

—CH₂—CH₂—CH₂—O—CH₃;

—CH₂—CH₂—CH₂—O—CH₂—CH₃;

—CH₂—CH₂—CH₂—O—[CH₂—CH₂—CH₂—O]ₙ₅—CH₃; and

—CH₂—CH₂—CH₂—O—[CH₂—CH₂—CH₂—O]ₙ₅—CH₂—CH₃;

n1 is an integer independently selected in each instance from the group of 1, 2, 3, and 4;

n2 is an integer independently selected in each instance from the group of 1, 2, 3, 4, 5, 6, 7, 8, 9, and 10;

n3 is an integer independently selected in each instance from the group of 1, 2, 3, 4, 5, 6, 7, 8, 9, and 10;

n4 is an integer independently selected in each instance from the group of 1, 2, 3, 4, 5, 6, 7, 8, 9, and 10; and

n5 is an integer independently selected in each instance from the group of 1, 2, 3, 4, 5, 6, 7, 8, 9, and 10;

with the proviso that the sum of n2+n4 is not greater than 10;

with the proviso that the sum of n3+n5 is not greater than 10;

with the proviso that the sum of n2+n5 is not greater than 10; and

with the proviso that the sum of n3+n4 is not greater than 10.

Another embodiment provides a compound of compound of Formula (II):

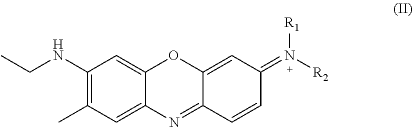

wherein:

R₁ is selected from the group of ethyl, —(CH₂)ₙ₁—SO₃⁻, —(CH₂)ₙ₁—N⁺(CH₃)₃;

—CH₂—CH₂—O—CH₃;

—CH₂—CH₂—O—CH₂—CH₃,

—CH₂—CH₂—O—[CH₂—CH₂—O]ₙ₂—CH₃;

—CH₂—CH₂—O—[CH₂—CH₂—O]ₙ₂—CH₂—CH₃;

—CH₂—CH₂—CH₂—O—CH₃;

—CH₂—CH₂—CH₂—O—CH₂—CH₃;

—CH₂—CH₂—CH₂—O—[CH₂—CH₂—CH₂—O]ₙ₃—CH₃; and

—CH₂—CH₂—CH₂—O—[CH₂—CH₂—CH₂—O]ₙ₃—CH₂—CH₃;

R₂ is selected from the group of —(CH₂)ₙ₁—SO₃⁻, —(CH₂)ₙ₁—N⁺(CH₃)₃;

—CH₂—CH₂—O—CH₃;

—CH₂—CH₂—O—CH₂—CH₃,

—CH₂—CH₂—O—[CH₂—CH₂—O]ₙ₄—CH₃;

—CH₂—CH₂—O—[CH₂—CH₂—O]ₙ₄—CH₂—CH₃;

—CH₂—CH₂—CH₂—O—CH₃;

—CH₂—CH₂—CH₂—O—CH₂—CH₃;

—CH₂—CH₂—CH₂—O—[CH₂—CH₂—CH₂—O]ₙ₅—CH₃; and

—CH₂—CH₂—CH₂—O—[CH₂—CH₂—CH₂—O]ₙ₅—CH₂—CH₃;

n1 is an integer independently selected in each instance from the group of 1, 2, 3, and 4; n2 is an integer independently selected in each instance from the group of 1, 2, 3, 4, 5, 6, 7, and 8;

n3 is an integer independently selected in each instance from the group of 1, 2, 3, 4, 5, 6, 7, and 8;

n4 is an integer independently selected in each instance from the group of 1, 2, 3, 4, 5, 6, 7, and 8; and

n5 is an integer independently selected in each instance from the group of 1, 2, 3, 4, 5, 6, 7, and 8;

with the proviso that the sum of n2+n4 is not greater than 10;

with the proviso that the sum of n3+n5 is not greater than 10;

with the proviso that the sum of n2+n5 is not greater than 10; and

with the proviso that the sum of n3+n4 is not greater than 10.

A further embodiment provides a compound of Formula (II), wherein R₁, R₂ and n1 are as defined above; and

n2 is an integer independently selected in each instance from the group of 1, 2, 3, 4, 5, and 6;

n3 is an integer independently selected in each instance from the group of 1, 2, 3, 4, 5, and 6;

n4 is an integer independently selected in each instance from the group of 1, 2, 3, 4, 5, and 6; and

n5 is an integer independently selected in each instance from the group of 1, 2, 3, 4, 5, and 6;

with the proviso that the sum of n2+n4 is not greater than 10; and

with the proviso that the sum of n3+n5 is not greater than 10;

with the proviso that the sum of n2+n5 is not greater than 10; and

with the proviso that the sum of n3+n4 is not greater than 10.

Another embodiment provides a compound of Formula (II), wherein R₁, R₂ and n1 are as defined above; and

n2 is an integer independently selected in each instance from the group of 1, 2, 3, 4, 5, and 6;

n3 is an integer independently selected in each instance from the group of 1, 2, 3, 4, 5, and 6;

n4 is an integer independently selected in each instance from the group of 1, 2, 3, 4, 5, and 6; and

n5 is an integer independently selected in each instance from the group of 1, 2, 3, 4, 5, and 6;

with the proviso that the sum of n2+n4 is not greater than 8; and

with the proviso that the sum of n3+n5 is not greater than 8;

with the proviso that the sum of n2+n5 is not greater than 8; and

with the proviso that the sum of n3+n4 is not greater than 8.

Another embodiment provides a compound of Formula (II), wherein R₁, R₂ and n1 are as defined above; and

n2 is an integer independently selected in each instance from the group of 1, 2, 3, and 4;

n3 is an integer independently selected in each instance from the group of 1, 2, 3, and 4;

n4 is an integer independently selected in each instance from the group of 1, 2, 3, and 4; and

n5 is an integer independently selected in each instance from the group of 1, 2, 3, and 4;

with the proviso that the sum of n2+n4 is not greater than 6; and

with the proviso that the sum of n3+n5 is not greater than 6;

with the proviso that the sum of n2+n5 is not greater than 6; and

with the proviso that the sum of n3+n4 is not greater than 6.

Another embodiment provides a compound of Formula (II), wherein R₁, R₂ and n1 are as defined above; and

n2 is an integer independently selected in each instance from 1, 2, and 3;

n3 is an integer independently selected in each instance from 1, 2, and 3;

n4 is an integer independently selected in each instance from 1, 2, and 3; and

n5 is an integer independently selected in each instance from 1, 2, and 3;

with the proviso that the sum of n2+n4 is not greater than 4; and

with the proviso that the sum of n3+n5 is not greater than 4;

with the proviso that the sum of n2+n5 is not greater than 4; and

with the proviso that the sum of n3+n4 is not greater than 4.

For each separate embodiment concerning a compound of Formula (II), above, there is another embodiment in which R₂, X₁, n1, n2, n3, n4, n5, and the provisos are as defined for the specific embodiment in question and R₁ is selected from the group of methyl, ethyl, n-propyl, and isopropyl.

For each separate embodiment concerning a compound of Formula (II), above, there is still another embodiment in which R₂, X₁, n1, n2, n3, n4, n5, and the provisos are as defined for the specific embodiment in question and R₁ is selected from the group of methyl, ethyl, and n-propyl.

For each separate embodiment concerning a compound of Formula (II), above, there is still another embodiment in which R₂, X₁, n1, n2, n3, n4, n5, and the provisos are as defined for the specific embodiment in question and R₁ is ethyl.

Further provided is a compound of Formula (III)

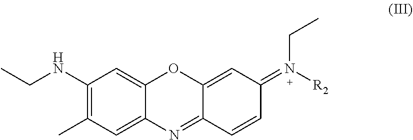

wherein:

R₂ is selected from the group of —(CH₂)ₙ₁—SO₃⁻, —(CH₂)ₙ₁—N⁺(CH₃)₃;

—CH₂—CH₂—O—CH₃;

—CH₂—CH₂—O—CH₂—CH₃,

—CH₂—CH₂—O—[CH₂—CH₂—O]ₙ₂—CH₃;

—CH₂—CH₂—O—[CH₂—CH₂—O]ₙ₂—CH₂—CH₃;

—CH₂—CH₂—CH₂—O—CH₃;

—CH₂—CH₂—CH₂—O—CH₂—CH₃;

—CH₂—CH₂—CH₂—O—[CH₂—CH₂—CH₂—O]ₙ₃—CH₃; and

—CH₂—CH₂—CH₂—O—[CH₂—CH₂—CH₂—O]ₙ₃—CH₂—CH₃;

n1 is an integer independently selected in each instance from the group of 1, 2, 3, and 4;

n4 is an integer independently selected in each instance from the group of 1, 2, 3, 4, 5, 6, 7, and 8; and

n5 is an integer independently selected in each instance from the group of 1, 2, 3, 4, 5, 6, 7, and 8;

Another embodiment comprises a compound of Formula (III), wherein:

R₂ is selected from the group of —(CH₂)ₙ₁—SO₃⁻, —(CH₂)ₙ₁—N⁺(CH₃)₃; —CH₂—CH₂—O—CH₃; —CH₂—CH₂—O—CH₂—CH₃; —CH₂—CH₂—O—[CH₂—CH₂—O]ₙ₂—CH₃; —CH₂—CH₂—O—[CH₂—CH₂—O]ₙ₂—CH₂—CH₃; —CH₂—CH₂—CH₂—O—CH₃; —CH₂—CH₂—CH₂—O—CH₂—CH₃; —CH₂—CH₂—CH₂—O—[CH₂—CH₂—CH₂—O]ₙ₃—CH₃; and —CH₂—CH₂—CH₂—O—[CH₂—CH₂—CH₂—O]ₙ₃—CH₂—CH₃;

n1 is an integer independently selected in each instance from the group of 1, 2, 3, and 4;

n4 is an integer independently selected in each instance from the group of 1, 2, 3, 4, 5, and 6; and

n5 is an integer independently selected in each instance from the group of 1, 2, 3, 4, 5, and 6.

Yet another embodiment comprises a compound of Formula (III), wherein:

R₂ is selected from the group of —(CH₂)ₙ₁—SO₃⁻, —(CH₂)ₙ₁—N⁺(CH₃)₃; —CH₂—CH₂—O—CH₃; —CH₂—CH₂—O—CH₂—CH₃; —CH₂—CH₂—O—[CH₂—CH₂—O]ₙ₂—CH₃; —CH₂—CH₂—O—[CH₂—CH₂—O]ₙ₂—CH₂—CH₃; —CH₂—CH₂—CH₂—O—CH₃; —CH₂—CH₂—CH₂—O—CH₂—CH₃; —CH₂—CH₂—CH₂—O—[CH₂—CH₂—CH₂—O]ₙ₃—CH₃; and —CH₂—CH₂—CH₂—O—[CH₂—CH₂—CH₂—O]ₙ₃—CH₂—CH₃;

n1 is an integer independently selected in each instance from the group of 1, 2, 3, and 4;

n4 is an integer independently selected in each instance from the group of 1, 2, 3, and 4; and

n5 is an integer independently selected in each instance from the group of 1, 2, 3, and 4.

Further provided is a compound of Formula (IV):

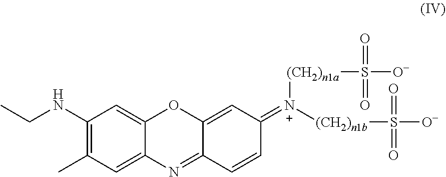

wherein n1a and n1b are each integers independently selected from the group of 1, 2, 3, and 4.

Provided is a compound of Formula (V):

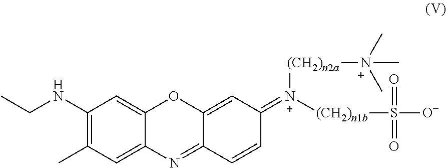

wherein n2a and n1b are each integers independently selected from the group of 1, 2, 3, and 4.

Also provided is a compound of Formula (VI):

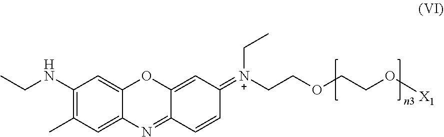

wherein X₁ is selected from the group of C₁-C₆ straight or branched alkyl, C₂-C₆ straight or branched alkenyl, C₁-C₆ straight or branched alkynyl, and —Si(C₁-C₄ alkyl)₃; and

n3 is an integer selected from the group of 1, 2, 3, 4, 5, 6, 7, 8, 9, and 10.

Another embodiment comprises a compound of Formula (VI), wherein X₁ is selected from the group of C₁-C₄ straight or branched alkyl, C₂-C₄ straight or branched alkenyl, C₁-C₄ straight or branched alkynyl, and —Si(C₁-C₄ alkyl)₃; and

n3 is an integer selected from the group of 1, 2, 3, 4, 5, 6, 7, 8, 9, and 10.

Another embodiment comprises a compound of Formula (VI), wherein X₁ is selected from the group of C₁-C₃ alkyl, C₂-C₃ alkenyl, or C₂-C₃ alkynyl, and —Si(C₁-C₃ alkyl)₃; and

n3 is an integer selected from the group of 1, 2, 3, 4, 5, 6, 7, 8, 9, and 10.

Another embodiment comprises a compound of Formula (VI), wherein X₁ is selected from the group of C₁-C₂ alkyl, ethenyl, or ethynyl, and —Si(C₁-C₂ alkyl)₃; and

n3 is an integer selected from the group of 1, 2, 3, 4, 5, 6, 7, 8, 9, and 10.

Another embodiment comprises a compound of Formula (VI), wherein X₁ is C₁-C₂ alkyl; and n3 is an integer selected from the group of 1, 2, 3, 4, 5, 6, 7, 8, 9, and 10.

Another embodiment comprises a compound of Formula (VI), wherein X₁ is selected from the group of C₁-C₄ straight or branched alkyl, C₂-C₄ straight or branched alkenyl, C₁-C₄ straight or branched alkynyl, and —Si(C₁-C₄ alkyl)₃; and n3 is an integer selected from the group of 1, 2, 3, 4, 5, 6, 7, and 8.

Another embodiment comprises a compound of Formula (VI), wherein X₁ is selected from the group of C₁-C₃ straight or branched alkyl, C₂-C₃ straight or branched alkenyl, C₁-C₃ straight or branched alkynyl, and —Si(C₁-C₃ alkyl)₃; and

n3 is an integer selected from the group of 1, 2, 3, 4, 5, 6, 7, and 8.

Another embodiment comprises a compound of Formula (VI), wherein X₁ is selected from the group of C₁-C₂ alkyl, ethenyl, or ethynyl, and —Si(C₁-C₂ alkyl)₃; and

n3 is an integer selected from the group of 1, 2, 3, 4, 5, 6, 7, and 8.

Another embodiment comprises a compound of Formula (VI), wherein X₁ is C₁-C₂ alkyl; and n3 is an integer selected from the group of 1, 2, 3, 4, 5, 6, 7, and 8.

Another embodiment comprises a compound of Formula (VI), wherein X₁ is selected from the group of C₁-C₄ straight or branched alkyl, C₂-C₄ straight or branched alkenyl, C₁-C₄ straight or branched alkynyl, and —Si(C₁-C₄ alkyl)₃; and n3 is an integer selected from the group of 1, 2, 3, 4, 5, and 6.

Another embodiment comprises a compound of Formula (VI), wherein X₁ is selected from the group of C₁-C₃ straight or branched alkyl, C₂-C₃ straight or branched alkenyl, C₁-C₃ straight or branched alkynyl, and —Si(C₁-C₃ alkyl)₃; and

n3 is an integer selected from the group of 1, 2, 3, 4, 5, and 6.

Another embodiment comprises a compound of Formula (VI), wherein X₁ is selected from the group of C₁-C₂ alkyl, ethenyl, or ethynyl, and —Si(C₁-C₂ alkyl)₃; and

n3 is an integer selected from the group of 1, 2, 3, 4, 5, and 6.

Another embodiment comprises a compound of Formula (VI), wherein X₁ is C₁-C₂ alkyl; and n3 is an integer selected from the group of 1, 2, 3, 4, 5, and 6.

Another embodiment comprises a compound of Formula (VI), wherein X₁ is selected from the group of C₁-C₄ straight or branched alkyl, C₂-C₄ straight or branched alkenyl, C₁-C₄ straight or branched alkynyl, and —Si(C₁-C₄ alkyl)₃; and n3 is an integer selected from the group of 1, 2, 3, and 4.

Another embodiment comprises a compound of Formula (VI), wherein X₁ is selected from the group of C₁-C₃ straight or branched alkyl, C₂-C₃ straight or branched alkenyl, C₁-C₃ straight or branched alkynyl, and —Si(C₁-C₃ alkyl)₃; and

n3 is an integer selected from the group of 1, 2, 3, and 4.

Another embodiment comprises a compound of Formula (VI), wherein X₁ is selected from the group of C₁-C₂ alkyl, ethenyl, or ethynyl, and —Si(C₁-C₂ alkyl)₃; and

n3 is an integer selected from the group of 1, 2, 3, and 4.

Another embodiment comprises a compound of Formula (VI), wherein X₁ is selected from the group of C₁-C₄ straight or branched alkyl, C₂-C₄ straight or branched alkenyl, C₁-C₄ straight or branched alkynyl, and —Si(C₁-C₄ alkyl)₃; and n3 is an integer selected from the group of 2, 3, and 4.

Another embodiment comprises a compound of Formula (VI), wherein X₁ is selected from the group of C₁-C₄ straight or branched alkyl, C₂-C₄ straight or branched alkenyl, C₁-C₄ straight or branched alkynyl, and —Si(C₁-C₄ alkyl)₃; and

n3 is an integer selected from the group of 2, 3, and 4.

Another embodiment comprises a compound of Formula (VI), wherein X₁ is selected from the group of C₁-C₂ alkyl, ethenyl, or ethynyl, and —Si(C₁-C₂ alkyl)₃; and

n3 is an integer selected from the group of 2, 3, and 4.

Another embodiment comprises a compound of Formula (VI), wherein X₁ is C₁-C₂ alkyl; and n3 is an integer selected from the group of 2, 3, and 4.

Provided is a compound of Formula (VII):

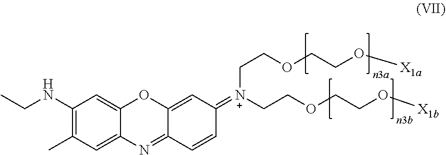

wherein X₁ₐ and X1b are selected independently from the group of C₁-C₆ straight or branched alkyl, C₂-C₆ straight or branched alkenyl, C₁-C₆ straight or branched alkynyl, and —Si(C₁-C₄ alkyl)₃; and

n3a and n3b are each an integer independently selected from the group of 1, 2, 3, 4, 5, 6, 7, 8, 9, and 10; with the proviso that the sum of n3a+n3b is not greater than 10.

Another embodiment provides a compound of Formula (VII), wherein X₁ₐ and X_{1b} are selected independently from the group of C₁-C₄ straight or branched alkyl, C₂-C₄ straight or branched alkenyl, C₁-C₄ straight or branched alkynyl, and —Si(C₁-C₄ alkyl)₃; and n3a and n3b are each an integer independently selected from the group of 1, 2, 3, 4, 5, 6, and 7; with the proviso that the sum of n3a+n3b is not greater than 8.

Another embodiment provides a compound of Formula (VII), wherein X₁ₐ and X_{1b} are selected independently from the group of C₁-C₃ straight or branched alkyl, C₂-C₃ straight or branched alkenyl, C₁-C₃ straight or branched alkynyl, and —Si(C₁-C₃ alkyl)₃; and

n3a and n3b are each an integer independently selected from the group of 1, 2, 3, 4, 5, 6, and 7; with the proviso that the sum of n3a+n3b is not greater than 8.

Another embodiment provides a compound of Formula (VII), wherein X₁ₐ and X_{1b} are selected independently from the group of C₁-C₂ alkyl, ethenyl, or ethynyl, and —Si(C₁-C₂ alkyl)₃; and n3a and n3b are each an integer independently selected from the group of 1, 2, 3, 4, 5, 6, and 7; with the proviso that the sum of n3a+n3b is not greater than 8.

Another embodiment provides a compound of Formula (VII), wherein X₁ₐ and X_{1b} are selected independently from the group of methyl and ethyl; and

n3a and n3b are each an integer independently selected from the group of 1, 2, 3, 4, 5, 6, and 7; with the proviso that the sum of n3a+n3b is not greater than 8.

Another embodiment provides a compound of Formula (VII), wherein X₁ₐ and X_{1b} are selected independently from the group of C₁-C₄ straight or branched alkyl, C₂-C₄ straight or branched alkenyl, C₁-C₄ straight or branched alkynyl, and —Si(C₁-C₄ alkyl)₃; and

n3a and n3b are each an integer independently selected from the group of 1, 2, 3, 4, and 5; with the proviso that the sum of n3a+n3b is not greater than 6.

Another embodiment provides a compound of Formula (VII), wherein X₁ₐ and X_{1b} are selected independently from the group of C₁-C₃ straight or branched alkyl, C₂-C₃ straight or branched alkenyl, C₁-C₃ straight or branched alkynyl, and —Si(C₁-C₃ alkyl)₃; and

n3a and n3b are each an integer independently selected from the group of 1, 2, 3, 4, and 5; with the proviso that the sum of n3a+n3b is not greater than 6.

Another embodiment provides a compound of Formula (VII), wherein X₁ₐ and X_{1b} are selected independently from the group of C₁-C₂ alkyl, ethenyl, or ethynyl, and —Si(C₁-C₂ alkyl)₃; and n3a and n3b are each an integer independently selected from the group of 1, 2, 3, 4, and 5; with the proviso that the sum of n3a+n3b is not greater than 6.

Another embodiment provides a compound of Formula (VII), wherein X₁ₐ and X_{1b} are selected independently from methyl and ethyl; and n3a and n3b are each an integer independently selected from the group of 1, 2, 3, 4, and 5; with the proviso that the sum of n3a+n3b is not greater than 6.

Another embodiment provides a compound of Formula (VII), wherein X₁ₐ and X_{1b} are selected independently from the group of C₁-C₄ straight or branched alkyl, C₂-C₄ straight or branched alkenyl, C₁-C₄ straight or branched alkynyl, and —Si(C₁-C₄ alkyl)₃; and

n3a and n3b are each an integer independently selected from the group of 1, 2, and 3; with the proviso that the sum of n3a+n3b is not greater than 4.

Another embodiment provides a compound of Formula (VII), wherein X₁ₐ and X_{1b} are selected independently from the group of C₁-C₃ straight or branched alkyl, C₂-C₃ straight or branched alkenyl, C₁-C₃ straight or branched alkynyl, and —Si(C₁-C₃ alkyl)₃; and

n3a and n3b are each an integer independently selected from the group of 1, 2, and 3; with the proviso that the sum of n3a+n3b is not greater than 4.

Another embodiment provides a compound of Formula (VII), wherein X₁ₐ and X_{1b} are selected independently from the group of C₁-C₂ alkyl, ethenyl, or ethynyl, and —Si(C₁-C₂ alkyl)₃; and

n3a and n3b are each an integer independently selected from the group of 1, 2, and 3; with the proviso that the sum of n3a+n3b is not greater than 4.

Another embodiment provides a compound of Formula (VII), wherein X₁ₐ and X_{1b} are selected independently from the group of methyl and ethyl; and

n3a and n3b are each an integer independently selected from the group of 1, 2, and 3; with the proviso that the sum of n3a+n3b is not greater than 4.

### Definitions

A “subject” or a “patient” refers to any animal. The animal may be a mammal. Examples of suitable mammals include human and non-human primates, dogs, cats, sheep, cows, pigs, horses, mice, rats, rabbits, and guinea pigs. In some embodiments the subject or patient is a human, particularly including a human undergoing or in need of a surgical procedure or examination.

The term “nerve” used herein means a bundle of neural axons. Within a nerve, each axon is surrounded by a layer of connective tissue called the endoneurium. The axons are bundled together into groups called fascicles, and each fascicle is wrapped in a layer of connective tissue called the perineurium. The entire nerve is wrapped in a layer of connective tissue called the epineurium. The term “nerve” is intended to include any tissues (e.g., the sinoatrial node or the atriventricular node) or structures associated therewith (e.g., neuromuscular junctions).

The term “nerve-specific” or “nerve specific” herein refers to an agent that is drawn to a nerve or nerve tissue and may be used in fluorescent imaging techniques to help contrast and differentiate the nerve or nerve tissue from surrounding cells and/or tissues. The term “nerve specificity” refers to the nature or activity of an agent being nerve-specific.

The term “near infrared” or the acronym “(NIR)” refers to light at the near infrared spectrum, generally at a wavelength of about 0.65 to about 1.4 μm (700 nm-1400 nm. It may also refer to a range designated by the International Organization for Standardization as from a wavelength of about 0.78 μm to about 3 μm. In some embodiments, the preferred near infrared spectroscopy and imaging (NIRS) range is from about 650 nm to about 950 nm. In other embodiments, the preferred near infrared spectroscopy and imaging (NIRS) range is from about 650 nm to about 900 nm.

In some embodiments the agents and/or compositions comprising them are intended for direct/topical administration. Direct or topical administration are understood herein to comprise the administration of an agent or composition directly to surface of a tissue, organ, nerve bundle, or other bodily component. In some methods, the administration may be accomplished by brushing, spraying, or irrigation with the appropriate compound or composition.

In other embodiments, the agents and/or compositions may be administered systemically to the patient or subject, such as through intravenous injection or infusion.

In other embodiments, the agents and/or compositions may be administered locally to a desired tissue or organ, such as through injection.

The terms “effective amount” or “medically effective amount” or like terms refers to an amount of a compound or composition as described herein to cover a target area sufficiently to complete binding to one or more nerves such that they may be identified through relevant imaging techniques, particularly near-infrared imaging techniques.

The term “imaging” herein refers to the use of fluorescent compounds in conventional medical imaging techniques including, but not limited to, those related to fluorescence image-guided surgery (including minimally invasive laparoscopy or endoscopy techniques), computer-assisted surgery or surgical navigation, radiosurgery or radiation therapy, interventional radiology, fluorescence microscopy, and laser-confocal microscopy. These techniques may include near infrared wavelengths from about 650 nm to 900 nm.

The term “label” refers to a molecule that facilitates the visualization and/or detection of a targeting molecule disclosed herein. In some embodiments, the label is a fluorescent moiety. The term “labeling” refers to a successful administration of the label to a target to allow such detection.

As used herein, the terms “robotic surgery”, “robot-assisted surgery”, or “computer-assisted surgery” refer to surgical techniques involving robotic systems that control the movement of medical instruments to conduct a surgical procedure with precise, flexible, and/or minimally invasive actions designed to limit the amount of surgical trauma, blood loss, pain, scarring, and post-surgical patient recovery time and/or complications, such as infection at the surgical area. Examples of robotic surgery include those conducted using the da Vinci Surgical System (Intuitive Surgical, Sunnyvale, Calif., USA) approved by the U.S. Food and Drug Administration in 2000.

The terms “surgery” or “surgical method” as used herein, refers to any method used to manipulate, change, or cause an effect by a physical intervention. These methods include, but are not limited to open surgery, endoscopic surgery, laparoscopic surgery, minimally invasive surgery, robotic surgery, any procedures that may affect any neuron or nerve, such as placement of retractors during spinal surgery, electrically conducting cardiac tissue or nerve ablation, epidural injection, intrathecal injections, neuron or nerve blocks, implantation of devices such as neuron or nerve stimulators and implantation of pumps. These methods may also include biopsy or other invasive techniques for the collection of cell or tissue samples, such as for diagnostic purposes.

As used herein, the term “targeting molecule” refers to any agent (e.g., peptide, protein, nucleic acid polymer, aptamer, or small molecule) that associates with (e.g., binds to) a target of interest. The target of interest may be a nerve cell or an organ or tissue associated with one or more nerve cells or nerve structures. In some embodiments, the targeting molecule is any agent that associates with (e.g., binds to) a target comprising one or more neurons, nerves, or tissues or structures associated therewith, i.e. nerve tissues, nervous system tissues, nerve bundles, etc. It is understood that nerve and nerve-related targets include those associated with the brain and spinal cord of the central nervous system (CNS) and the nerves of the peripheral nervous system (PNS).

The term “prostatectomy” refers to a surgical technique to remove all or part of a subject's prostate gland. A “radical prostatectomy” concerns removal of a subject's entire prostate gland, along with surrounding tissues, often including the seminal vesicles and nearby lymph nodes.

The terms “orthopedic limb repair” or “orthopedic limb repair surgeries” refer to surgical techniques performed on the limb musculoskeletal system of a subject. These techniques include limb reconstruction surgeries, joint replacement procedures, revision joint surgery, debridement, bone fusions, tendon or ligament repair, internal fixation of bone, and osteotamies.

The term “fluorophore” herein refers to any one of the compounds described herein for use in imaging techniques, particularly for nerve imaging techniques. Each of the compounds described herein as the product of a specific synthesis or described in a generic description is considered fluorophore for methods, uses, and compositions.

The term “variable” or “variables” used in the generic descriptions and claims herein refer to the entities or moieties that may, in some instances, be chosen from a specified group. Such variables may include R, R₁, R₂, n1, n2, n3, n4, n5, X₁, and the like.

All ranges disclosed and/or claimed herein are inclusive of the recited endpoint and independently combinable (for example, the ranges of “from 2 to 10” and “2-10” are inclusive of the endpoints, 2 and 10, and all the intermediate values).

The term “intraoperatively” as used in describing methods or uses herein refers to an activity that occurs during a surgical procedure or in immediate preparation for such procedure.

The term “alkyl” refers to a straight or branched hydrocarbon. For example, an alkyl group can have 1 to 6 carbon atoms (i.e., C₁-C₆ alkyl), 1 to 4 carbon atoms (i.e., C₁-C₄ alkyl), or 1 to 3 carbon atoms (i.e., C₁-C₃ alkyl).

The term “alkenyl” refers to a straight or branched hydrocarbon with at least one site of unsaturation, i.e. a carbon-carbon, sp² double bond. For example, an alkenyl group can have 2 to 6 carbon atoms (i.e., C₂-C₆ alkenyl) or 2 to 4 carbon atoms (i.e., C₂-C₄ alkenyl). Examples of suitable C₂-C₄ alkenyl groups include, but are not limited to, ethenyl or vinyl (—CH═CH₂), allyl (—CH₂CH═CH₂), but-1-enyl —CH═CH—CH₂—CH₃), but-2-enyl (CH₂—CH═CH—CH₃), but-3-enyl (—CH₂—CH₂—CH═CH).

### Methods of Use

Provided is a method of detecting nerves in a tissue or organ, the method comprising
a) administering an effective amount of a composition comprising a fluorophore as described herein to the tissue or organ to form a stained tissue or a stained organ; and
b) imaging the stained tissue or stained organ, thereby detecting nerves intraoperatively in the stained tissue or stained organ.

Provided is a method of detecting nerves intraoperatively in a subject undergoing surgery, the method comprising:
c) administering an effective amount of a composition comprising a fluorophore as described herein to the subject before or during surgery to form a stained tissue; and
d) imaging the stained tissue undergoing surgery in the subject, thereby detecting nerves intraoperatively in the subject undergoing surgery.

Also provided is a method of detecting nerves intraoperatively in a subject undergoing a prostatectomy surgery, the method comprising:
e) administering an effective amount of a composition comprising a fluorophore as described herein to the subject before or during the prostatectomy surgery to form a stained tissue; and
f) imaging the stained tissue undergoing surgery in the subject, thereby detecting nerves intraoperatively in the subject undergoing prostatectomy surgery.

In one embodiment is provided a method of detecting cavernous nerves intraoperatively in a subject undergoing a prostatectomy surgery, the method comprising:
g) administering an effective amount of a composition comprising a fluorophore as described herein to the subject before or during the prostatectomy surgery to form a stained tissue; and
h) imaging the stained tissue undergoing surgery in the subject, thereby detecting cavernous nerves intraoperatively in the subject undergoing prostatectomy surgery.

For each of the methods herein concerning a prostatectomy surgery or procedure, there is another embodiment in which the surgery or procedure is a radical prostatectomy.

For each of the methods above and herein, there is an embodiment in which the composition comprising a fluorophore is administered to the subject systemically.

For each of the methods above and herein, there is an embodiment in which the composition comprising a fluorophore is administered to the subject directly or topically, i.e. through direct administration or topical administration.

Within each of the methods herein, there is a further embodiment in which the administration of an effective amount of a composition comprising a fluorophore as described herein to the subject before or during the prostatectomy surgery to form a stained tissue can be completed in fifteen minutes or less. In a still further embodiment, the administration of an effective amount of a composition comprising a fluorophore as described herein to the subject before or during the prostatectomy surgery to form a stained tissue can be completed in ten minutes or less.

Also provided herein are methods of imaging nervous tissue tumors (neoplasms), including Gliomas, such as bliomatosis cerbri, Oligoastrocytomas, Choroid plexus papillomas, Ependymomas, Astrocytomas (Pilocytic astrocytomas and Glioblastoma multiforme), Dysembryoplastic neuroepithelial tumors, Oligodendrogliomas, Medulloblastomas, and Primitive neuroectodermal tumors; Neuroepitheliomatous tumors, such as Ganglioneuromas, Neruoblastomas, Atypical teratoid rhabdoid tumors, Retinoblastomas, and Esthesioneuroblastomas; and Nerve Sheath Tumors, such as Neruofibromas (Neurofibrosarcomas and Neurofibromatosis), Schannomas, Neurinomas, Acoustic neuromas, and Neuromas.

Provided is a method of imaging a target area in a subject, the method comprising contacting the target area in the subject with a compound selected from those herein and detecting the compound in the target using fluorescence or near-infrared imaging.

Also provided is a method of imaging one or more nerves in a target area in a subject, the method comprising contacting the target area in the subject with a compound selected from those herein and detecting the compound in the target using fluorescence imaging.

Also provided is a method of imaging one or more nerves in a target area in a subject, the method comprising contacting the target area in the subject with a compound selected from those herein and detecting the compound in the target using near-infrared imaging.

Also provided is a method of minimizing nerve damage in a target area in a subject during a medical procedure, the method comprising the steps of:
a) contacting the target area in the subject with a compound selected from those herein;
b) detecting one or more nerves bound by the compound in the target area using fluorescence imaging; and
c) minimizing actions of the medical procedure that may damage one or more nerves detected.

The method above may be used to identify nerves and minimize damage to them that may be caused by a medical procedure, including traumatic, thermal, and radiological damage or that are caused by the application of therapeutic agents, anesthetics, or anesthesia in the target area.

In some embodiments, the medical procedure referenced in the method above is a surgical procedure. In other embodiments, the medical procedure is a biopsy procedure, a radiological procedure, or the application of anesthetic or anesthesia to the subject. In further embodiments, the medical procedure in the method above is the insertion or implantation of a medical device, including a medical pump, stent, pacemaker, port, artificial joints, valves, screws, pins, plates, rods, cosmetic implants, neurostimulators, and the like.

Also provided is the use of any compound disclosed herein in the preparation of a composition for use in imaging one or more nerves in a subject using from near-infrared imaging.

Nerve damage plagues surgical outcomes, significantly affecting post-surgical quality of life. Despite the practice of nerve sparing techniques for decades, intraoperative nerve identification and sparing remains difficult and success rates are strongly correlated with surgeon experience level and ability to master the technique (Walsh & Donker. The Journal of urology 128, 492-497 (1982); Ficarra et al. Eur Urol 62, 405-417 (2012); Damber & Khatami. Acta oncologica 44, 599-604 (2005)). Fluorescence-guided surgery (FGS) shows promise for enhanced visualization of specifically highlighted tissue, such as nerves and tumor tissue, intraoperatively. FGS using optical imaging technology is capable of real-time, wide field identification of targeted tissues with high sensitivity and specificity from tissue targeted fluorescent probes. See, for instance: Frangioni. Journal of clinical oncology: official journal of the American Society of Clinical Oncology 26, 4012-4021 (2008); Gibbs. Quantitative imaging in medicine and surgery 2, 177-187 (2012); Gioux et al. Molecular imaging 9, 237-255 (2010); Vahrmeijer et al. Nature reviews. Clinical oncology 10, 507-518 (2013); and Nguyen et al. Nature reviews. Cancer 13, 653-662 (2013). Operating in the near-infrared (NIR) optical window (650-900 nm wavelengths) where tissue chromophore absorbance, autofluorescence and scattering are minimal, FGS technologies have the ability to identify targeted tissues at millimeter to centimeter depths against a black background (Chance. Annals of the New York Academy of Sciences 838, 29-45 (1998); Gibbs. Quantitative imaging in medicine and surgery 2, 177-187 (2012)).

Several imaging systems have been developed for FGS applications. see, for instance: Lee et al. Plastic and reconstructive surgery 126, 1472-1481 (2010); Tummers et al. European journal of surgical oncology: the journal of the European Society of Surgical Oncology and the British Association of Surgical Oncology 40, 850-858 (2014); Troyan et al. Annals of surgical oncology 16, 2943-2952 (2009); Ashitate et al. Real-time simultaneous near-infrared fluorescence imaging of bile duct and arterial anatomy. The Journal of surgical research 176, 7-13 (2012); Verbeek et al. The Journal of urology 190, 574-579 (2013); Gibbs-Strauss et al. Molecular imaging 10, 91-101 (2011); Hirche et al. Surgical innovation 20, 516-523 (2013); Gotoh et al. Journal of surgical oncology 100, 75-79 (2009); and Kitagawa et al. Anticancer research 35, 6201-6205 (2015); Importantly, the da Vinci surgical robot, frequently used for robotic assisted radical prostatectomy (RP), can be equipped with an FDA approved fluorescence imaging channel.

Direct administration (also sometimes referred to as local administration) is an attractive alternative to systemic administration of fluorescent probes for minimizing potential toxicity and easing regulatory burdens for first in human clinical studies. By selectively labeling tissues within the surgical field, direct administration requires a significantly lower dose than systemic administration. A direct administration methodology has been developed that provides equivalent nerve signal to background (SBR) to systemic administration following a 15-minute staining protocol. Barth & Gibbs. Theranostics 7, 573-593 (2017). This methodology has been successfully applied to autonomic nerve models, which closely mimic the nerves surrounding the prostate. This method has additional benefits in the application to RP since nerve labeling via systemic administration during RP would generate high background from nerves in the prostate, which are not able to be spared, and renal fluorophore clearance, producing significant fluorescence signal in the urine within the adjacent bladder. Both of these extraneous fluorescence signals would diminish the ability to identify the cavernous nerves within the neurovascular bundle (NVB), which are responsible for continence and potency (Barth and Summer. Theranostics (2016). Tewari et al. BJU international 98, 314-323 (2006); Patel et al. Eur Urol 61, 571-576 (2012)). Perhaps most importantly, the direct administration methodology requires 16 times lower dose than systemic administration and when scaled to humans by body surface area the dose falls within the requirements for clinical translation under an exploratory investigational new drug (eIND) application to the FDA. Studies conducted under an eIND require minimal preclinical toxicity testing, since only a microdose (<100 μg) is administered to each patient, significantly reducing the cost of first-in-human studies.

While the direct administration methodology has provided high nerve specificity and SBR with a short staining protocol in preclinical rodent models (Barth & Gibbs. Theranostics 7, 573-593 (2017)), preliminary staining studies in large animal models generated significant background. To facilitate clinical translation, an improved formulation strategy that is FDA approved and facilitates increased application control for staining a variety of tissue surfaces, angles, and morphologies will be required.

Several classes of nerve specific fluorescence imaging probes have been studied preclinically for FGS. See, for instance: Gibbs-Strauss et al. Molecular imaging 10, 91-101 (2011); Wu et al. Journal of medicinal chemistry 51, 6682-6688 (2008); Wang et al. The journal of histochemistry and cytochemistry: official journal of the Histochemistry Society 58, 611-621 (2010); Gibbs et al. PloS one 8, e73493 (2013); Stankoff et al. Proceedings of the National Academy of Sciences of the United States of America 103, 9304-9309 (2006); Cotero et al. Molecular imaging and biology: MIB: the official publication of the Academy of Molecular Imaging 14, 708-717 (2012); Cotero et al. PloS one 10, e0130276 (2015); Bajaj et al. The journal of histochemistry and cytochemistry: official journal of the Histochemistry Society 61, 19-30 (2013); Gibbs-Strauss et al. Molecular imaging 9, 128-140 (2010); Meyers et al. The Journal of Neuroscience: the official journal of the Society for Neuroscience 23, 4054-4065 (2003); Wang et al. The Journal of neuroscience: the official journal of the Society for Neuroscience 31, 2382-2390 (2011); Park et al. Theranostics 4, 823-833 (2014). Of these, oxazine fluorophores (e.g., Oxazine 4) have demonstrated the most promise for clinical translation, with high nerve specificity following both direct and systemic administration. Comparative Example No. 1 (3-(diethyl-14-azaneylidene)-N-ethyl-8-methyl-3H-phenoxazin-7-amine) is a particularly promising compound and was chosen as the lead compound for advancement to clinical studies. Although Comparative Example No. 1 has been shown to demonstrate high nerve specificity and adequate fluorescence signal for real time imaging, previous studies have been conducted utilizing a co-solvent formulation as a vehicle for intravenous injection (Gibbs-Strauss et al. Molecular imaging 10, 91-101 (2011); Barth & Gibbs. Theranostics 7, 573-593 (2017)). The co-solvent formulation is only stable at room temperature for <30 minutes, cannot solubilize concentrations above 5 mg/mL, and requires the use of dimethyl sulfoxide and Kolliphor EL as solubilizing agents, which hampers clinical translation due to vehicle induced toxicity issues. Additionally, the co-solvent formulation is liquid based and thus not ideal for staining angled or vertical tissue surfaces. Therefore, a clinically viable formulation with FDA approval was needed for direct administration and intravenous injection of nerve-specific fluorescence for FGS.

Formulations comprising one or more of the compounds disclosed herein can be used to image nerves or nerve tissue. In particular embodiments, the formulations of the disclosure can be used to image nerves or nerve tissue in a subject. In particular embodiments, images of nerves can be obtained intraoperatively during FGS. In particular embodiments, the visualization of nerves during FGS allows surgery to be performed on tissue of interest while sparing nerves so as to reduce incidence of nerve injury during surgery. The area where surgery is performed or nearby regions can be surgically exposed. Surgery can be performed on organs, which include tissues such as nerve tissue, muscle tissue, and adipose tissue. The surgery can be laparoscopic, which is minimally invasive and includes the use of a thin, tubular device (laparoscope) that is inserted through a keyhole incision into a part of a subject's body, such as the abdomen or pelvis. The surgery can be assisted by a robot. Robot-assisted surgery can offer more precision, flexibility, and control, and is often associated with minimally invasive surgery.

In particular embodiments, the fluorophore concentration in a formulation that is directly applied to nerve tissue includes a concentration range of 40 to 300 μg/mL. In particular embodiments, the fluorophore concentration in a formulation for direct application includes 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, and 200 μg/mL. In particular embodiments, the fluorophore concentration in a formulation for direct application is 50 μg/mL. In particular embodiments, the fluorophore concentration in a formulation for direct application is 200 μg/mL.

A formulation of the disclosure can be systemically applied to a subject for imaging of nerves. In particular embodiments, systemic application of a formulation includes intravenous injection of the formulation into a subject.

A formulation that is directly applied to a tissue can be allowed to penetrate the tissue for a given amount of time after direct application. In particular embodiments, the formulation can be allowed to penetrate the tissue for 30 seconds to 15 minutes, for 1 to 10 minutes, for 1 to 5 minutes, for 1 minute, for 2 minutes, for 3 minutes, for 4 minutes, or for 5 minutes. In particular embodiments, the formulation can be allowed to penetrate the tissue for 1 to 2 minutes. A formulation that is systemically applied to a subject can be administered a sufficient time before imaging such that the formulation can reach the area to be imaged and is present in such area at the time of imaging. In particular embodiments, a formulation that is systemically applied to a subject can be administered a sufficient time prior to imaging to allow uptake of the formulation by tissue in the subject. In particular embodiments, the formulation may be administered up to or less than 30 minutes, 1 hour, 2 hours, 3 hours, 4 hours, 5 hours, 6 hours, 7 hours, or 8 hours before imaging. The amount of time required may depend on the nerve imaging application and the administration site. In particular embodiments, the formulation is administered no more than 30 minutes, 1 hour, 2 hours, 3 hours, or 4 hours before imaging. In particular embodiments, the formulation is administered no more than 2 hours before imaging.

Tissue stained by a formulation including a fluorophore by direct application can be washed with buffer prior to imaging of the stained tissue. Washing of tissue stained by a formulation including a fluorophore can include flushing the tissue with an appropriate buffer and removing the buffer. In particular embodiments, the stained tissue can be washed 1 to 18 times, 1 to 10 times, 1 to 6 times, 1 time, 2 times, 3 times, 4 times, 5 times, or 6 times, with wash buffer. In particular embodiments, the stained tissue can be washed 6 times. In particular embodiments, the wash buffer is phosphate-buffered saline (PBS). In particular embodiments, washing the stained tissue removes unbound fluorophore. In particular embodiments, washing the stained tissue increases the nerve signal intensity and/or the signal to background ratio (SBR) as compared to no washing of the stained tissue. In particular embodiments, washing the stained tissue resolubilizes the fluorophore and allows for further diffusion of the fluorophore into the nerve tissue.

Imaging a tissue stained by a formulation including a fluorophore includes applying light to tissue that has been stained with a formulation of the disclosure. The light can be at a wavelength sufficient to excite the fluorophore in the formulation to fluoresce. In particular embodiments, light to excite the fluorophore is at a wavelength in the near infrared spectra. In particular embodiments, the fluorophore of a formulation emits at a wavelength in the near infrared spectra. In particular embodiments, the near infrared spectra includes a wavelength of 700 to 900 nm.

Imaging a tissue stained by a formulation including a fluorophore includes obtaining fluorescence images of the stained tissue by optical imaging systems such as ones described in the Examples.

In particular embodiments, imaging a tissue includes observing fluorescence images of the stained tissue. The fluorescence images can include still images (whether printed or on screen), or real-time images on a video monitor. In particular embodiments, the individual images of nerves obtained by staining of the nerves with the present formulations can be used for diagnostic purposes and for documentation of nerve location. By observing the fluorescence images the surgical team can determine the absence or presence of a nerve in the image. The surgical team can thus use information about the presence/absence or location of one or more nerves to determine how they will perform the surgical procedure. For example, based on information obtained through the disclosed methods, the surgical team may decide to perform a surgical cut at a point in the tissue where they are less likely to inadvertently cut or surgically contact a particular nerve based on the perceived absence of a nerve in an area of the tissue.

The information obtained from the obtained image can aid in grafting the ends of the nerves if they are transected. In the event of transection, nerve grafts can be applied directly to the ends to facilitate sprouting of regenerative neural fibers. In this case, the light visible from the fluorescence of the ends of transected nerves provides a target to guide the anastomosis of the nerves by the nerve graft.

Formulations of the present disclosure to detect nerve tissue can also be provided as kits. Kits for detecting nerve tissue can include, in different containers: (i) a water-based formulation comprising a fluorophore, and (ii) one or more wash buffers. Kits can also include a notice in the form prescribed by a governmental agency regulating the manufacture, use, or sale of pharmaceuticals or biological products, which notice reflects approval by the agency of manufacture, use, or sale for human administration. The notice may state that the provided active ingredients can be administered to a subject. The kits can include further instructions for using the kit, for example, instructions regarding: directly applying the formulations to a tissue; washing to remove excess formulation; systemically administering the formulations to a subject; applying light for visualization of the fluorophores; capturing fluorescent images of the tissue; proper disposal of related waste; and the like. The instructions can be in the form of printed instructions provided within the kit or the instructions can be printed on a portion of the kit itself. Instructions may be in the form of a sheet, pamphlet, brochure, CD-ROM, or computer-readable device, or can provide directions to instructions at a remote location, such as a website. In particular embodiments, kits can also include some or all of the necessary laboratory and/or medical supplies needed to use the kit effectively, such as syringes, ampules, tubing, gloves, tubes, buffers, and the like. Variations in contents of any of the kits described herein can be made.

### General

All reagents were purchased from Sigma Aldrich, Fisher Scientific, or TCI. Unless otherwise indicated, all commercially available starting materials were used directly without further purification. Analytical TLC was performed on Millipore ready-to-use plates with silica gel 60 (F254, 32-63 μm). Purification was performed on a Biotage Isolera Flash System using pre-packed silica gel cartridges or on a reverse phase preparative HPLC (Agilent 1250 Infinity HPLC).

### LCMS Characterization

Mass-to-charge ratio and purity of the Oxazine compounds were characterized on an Agilent 6244 time-of-flight LCMS with diode array detector VL+. Sample (10 uL) was injected into a C₁₈ column (Poroshell 120, 4.6×50 mm, 2.7 micron), and eluted with a solvent system of A (H₂O, 0.1% FA) and B (MeCN, 01.% FA) at 0.4 mL/min, from A/B=90/10 to 5/95 over 10 min, maintained at A/B=5/95 for additional 5 min. Ions were detected in positive ion mode by setting the capillary voltage at 4 kV and gas temperature at 350° C.

### Nerve-Specificity Screening Using Direct/Topical Administration

Each compound was screened for its tissue-specificity using a previously published direct/topical administration strategy in murine brachial plexus and sciatic nerves.¹ Each compound from the Oxazine library was formulated in the previously utilized co-solvent formulation (10% DMSO, 5% Kolliphor, 65% serum and 20% phosphate buffered saline) at 125 μM. 100 μL of the formulated Oxazine were incubated on the exposed brachial plexus or sciatic nerve for 5 minutes. The fluorophore containing solution was removed and the area was irrigated with saline 18 times to remove any unbound fluorophore. Co-registered fluorescence and color images were collected of each stained area 30 minutes after Oxazine direct/topical administration using a custom built macroscopic imaging system with 620/60 nm excitation and 700/75 nm bandpass emission filters. Custom written MatLab code was used to analyze the tissue specific fluorescence where regions of interest were selected on the nerve, muscle and adipose tissue using the white light images. These regions of interest were then analyzed on the co-registered matched fluorescence images permitting assessment of the nerve to muscle and nerve to adipose ratios.

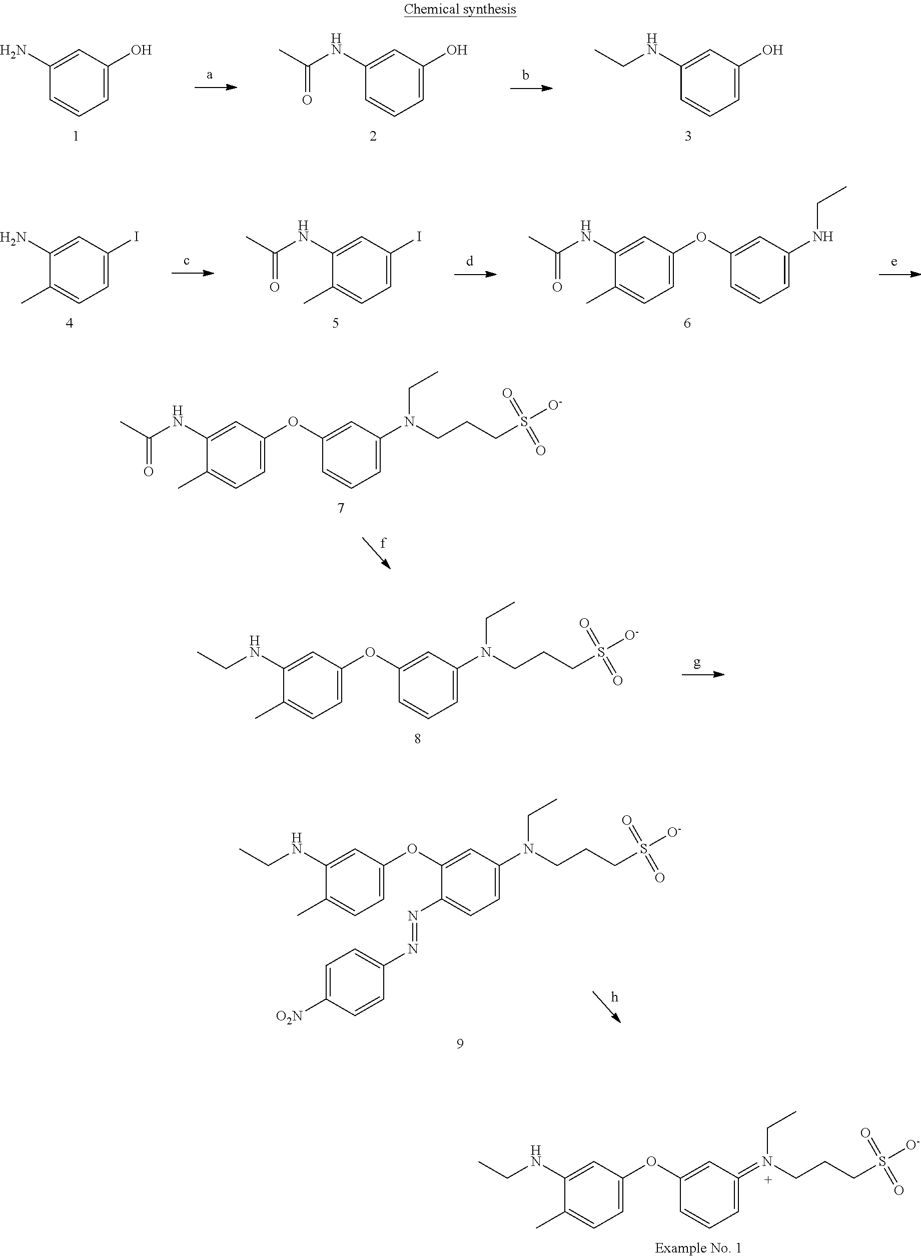

### Example No. 1—(E)-3-(ethyl(7-(ethylamino)-8-methyl-3H-phenoxazin-3-ylidene)ammonio)propane-1-sulfonate (LGW11-63)

Scheme 1: Synthetic route to Example 1. Reagents and conditions: a) Ac₂O, H₂O, 50° C. to room temperature (rt); b) BH₃-THF, THF, 0° C. to rt; c) Ac₂O, DMSO/H₂O( 1/9), 50° C. to rt; d) Compound 3, Cul, 2-picolinic acid, K₃PO₄, DMSO, 85° C.; e) 1,3-Propanesultone, Na₂CO₃, MeCN, 80° C.; f) BH₃-THF, THF, 0° C. to rt; g) I) 2M HCl, p-nitrobenzenediazonium tetrafluoroborate, 0° C.; II) Na₂CO₃, 0° C.; h) TsOH, EtOH, 80° C.

N-(3-hydroxyphenyl)acetamide (2): Compound 1 (1 g, 9.16 mmol) was suspended in 10 mL DI water, to which Acetic anhydride (2.60 mL, 27.49 mmol) was added dropwise. The reaction mixture was placed in an ultrasonication bath for 1 min, then was stirred in a water bath (50° C.) for 10 min. The resulting solution was stirred overnight at rt. After which, the solid was collected via vacuum filtration and washed with small portions of ice-cold DI water. The product was left in the funnel and air dried overnight to afford compound 2 (1.19 g, 86%) as a light gray solid, which was used for the next step without further purification.

3-(ethylamino)phenol (3): A solution of 2 (1 g, 6.62 mmol) in anhydrous THF (20 mL) was stirred in an ice bath under N₂ for 30 mins. Borane THF complex solution (1 M, 20 mL) was added to the solution above using a syringe pump over 30 mins, while maintaining the temperature of the solution below 5° C. The resulting reaction mixture was left in the ice bath and slowly warmed to rt. After 24 h, the solution was placed in an ice bath again, and excess borane reagent was destroyed by carefully adding MeOH until no gas evolved. The solvent was evaporated under reduced pressure, and the residue was purified by flash column chromatography with silica gel (25 g), using DCM/Hexane as eluent to obtain 3 (832 mg, 92%) as a solid.

N-(5-iodo-2-methylphenyl)acetamide (5): Compound 4 (2 g, 8.58 mmol) was dissolved in 2 mL DMSO, to which Acetic anhydride (2.43 mL, 25.75 mmol) was added dropwise. The reaction mixture was stirred in a water bath (50° C.) for 10 min, then stirred for additional 2 h at rt. 18 mL DI water was added to the reaction mixture, the resulting suspension was stirred overnight at rt. The solid was then collected via vacuum filtration and washed with small portions of DI water. The product was left in the funnel and air dried to afford compound 5 (2.09 g, 89%) as a light gray solid, which was used for the next step without further purification.

N-(5-(3-(ethylamino)phenoxy)-2-methylphenyl)acetamide (6): Compound 6 was synthesized using a slightly modified protocol published by Maiti and Buchwald.² An oven-dried microwave glass tube was charged with a magnetic stir bar, compound 3 (500 mg, 3.64 mmol), compound 5 (1.05 g, 3.83 mmol), Cul (69 mg, 0.36 mmol), 2-picolinic acid (90 mg, 0.73 mmol), and anhydrous K₃PO₄ (1.55 g, 7.29 mmol). The glass tube was evacuated under vacuum and backfilled 5 times with N₂ before the tube was immediately sealed with a Teflon cap. Anhydrous DMSO (5 mL) was delivered via a syringe. The reaction was then heated to 85° C. and stirred for 18 h. After cooling to rt, the reaction mixture was diluted with 50 mL DI water and extracted with DCM (4×50 mL). The combined organic layers were washed with brine and dried over anhydrous Na₂SO₄, then concentrated in vacuo. The residue was purified by flash column chromatography with silica gel (25 g), using DCM/Hexane as eluent to give compound 6 (871 mg, 74%) as an orange oil.

3-((3-(3-acetamido-4-methylphenoxy)phenyl)(ethyl)amino)propane-1-sulfonate (7): To a suspension of compound 6 (350 mg, 1.23 mmol) and Na₂CO₃ (261 mg, 2.46 mmol) in anhydrous MeCN (10 mL) under N₂, was added 1,3-Propanesultone (226 mg, 1.85 mmol) at rt. The reaction mixture was then heated up to 80° C., and stirred for 24 h. The solution was cooled down to rt, the solid was collected via vacuum filtration and washed with small portions of MeCN. The crude product was resuspended in EtOH (100 mL), and filtered through celite. The filtrate was concentrated to dryness by a rotary evaporator to give compound 7 (392 mg, 78%), and was used for the next step without further purification.

3-(ethyl(3-(3-(ethylamino)-4-methylphenoxy)phenyl)amino)propane-1-sulfonate (8): A solution of compound 7 (375 mg, 0.923 mmol) in anhydrous THF (10 mL) was stirred in an ice bath under N₂ for 30 mins. Borane tetrahydrofuran complex solution (1 M, 5 mL) was added to the solution above dropwise, while the temperature of the solution was maintained below 5° C. The resulting reaction mixture was left to stir in the ice bath and slowly warm to rt. After 24 h, the solution was placed in an ice bath again, and excess borane reagent was destroyed by carefully adding MeOH until no gas evolved. The solvent was evaporated under reduced pressure to give compound 8 (quantitative) and was used for the next step without further purification.

(E)-3-(ethyl(3-(3-(ethylamino)-4-methylphenoxy)-4-((4-nitrophenyl)diazenyl)phenyl)amino) propane-1-sulfonate (9): Compound 8 (100 mg, 0.255 mmol) was dissolved in MeOH (1 mL). The solution was chilled in an ice bath, then was treated with HCl (2 M, 5 mL). After 15 mins, p-nitrobenzenediazonium tetrafluoroborate (64 mg, 0.268 mmol) was added to the solution in 3 portions over 15 mins, then stirred at 0° C. for an additional 1 h. The solution was then carefully neutralized with solid Na₂CO₃ until the pH value of the solution had risen above 7. The crude product was deposited onto a C₁₈ cartridge, washed with DI water, air-dried, followed by elution with MeOH. The solvent was evaporated under reduced pressure to give compound 9 (quantitative) and was used for the next step without further purification.

(E)-3-(ethyl(7-(ethylamino)-8-methyl-3H-phenoxazin-3-ylidene)ammonio)propane-1-sulfonate (Example 1): Under N₂, compound 9 (50 mg, 0.092 mmol) and treated with p-toluenesulfonic acid monohydrate (53 mg, 0.277 mmol) were dissolved in ethanol (5 mL). The resulting solution was heated to 80° C., and stirred overnight. The solvent was evaporated under reduced pressure, and the residue was purified by reverse phase HPLC (MeCN/H₂O, 5-50%, linear gradient, TFA 0.1% as additive) to afford the compound of Example 1 (21 mg, 56%) as a dark blue solid.

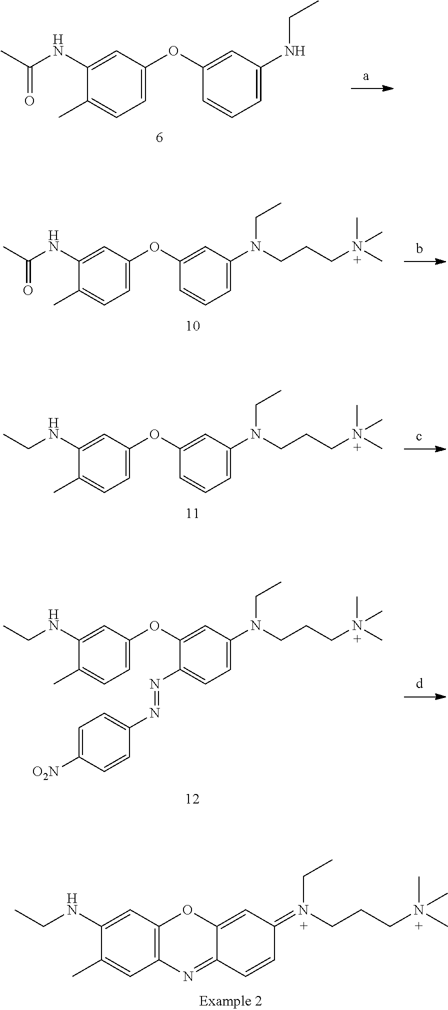

Example No. 2: (E)-N¹-ethyl-N¹-(7-(ethylamino)-8-methyl-3H-phenoxazin-3-ylidene)-N³,N³,N³-trimethylpropane-1,3-diaminium (LGW12-60) Scheme 2: Synthetic route to Example 2. Reagents and conditions: a) (3-Bromopropyl)trimethylammonium bromide, Na₂CO₃, MeCN, 80° C.; b) BH₃-THF, THF, 0° C. to rt; c) I) 2M HCl, p-nitrobenzenediazonium tetrafluoroborate, 0° C.; II) Na₂CO₃, 0° C.; d) TsOH, EtOH, 80° C.

3-((3-(3-acetamido-4-methylphenoxy)phenyl)(ethyl)amino)-N,N,N-trimethylpropan-1-aminium (10): Compound 6 (350 mg, 1.23 mmol), (3-Bromopropyl)trimethylammonium bromide (482 mg, 1.85 mmol), and Na₂CO₃ (261 mg, 2.46 mmol) were suspended in anhydrous MeCN (10 mL) under N₂. The reaction mixture was then heated up to 80° C., and stirred for 24 h. The solution was cooled down to rt, the solid was collected via vacuum filtration and washed with small portions of MeCN. The crude product was resuspended in EtOH (100 mL), and filtered through Celite. The filtrate was concentrated to dryness by a rotary evaporator to give compound 10 (quantitative) and was used for the next step without further purification.

3-(ethyl(3-(3-(ethylamino)-4-methylphenoxy)phenyl)amino)-N,N,N-trimethylpropan-1-aminium (11): A solution of compound 10 (350 mg, 0.91 mmol) in anhydrous THF (10 mL) was stirred in an ice bath under N₂ for 30 mins. Borane tetrahydrofuran complex solution (1 M, 10 mL) was added to the solution above dropwise, while the temperature of the solution was maintained below 5° C. The resulting reaction mixture was left to stir in the ice bath and slowly warm to rt. After 24 h, the solution was placed in an ice bath again, and excess borane reagent was destroyed by carefully adding MeOH until no gas evolved. The solvent was evaporated under reduced pressure to give compound 11 (quantitative) and was used for the next step without further purification.

(E)-3-(ethyl(3-(3-(ethylamino)-4-methylphenoxy)-4-((4-nitrophenyl)diazenyl)phenyl)amino)-N,N,N-trimethylpropan-1-aminium (12): Compound 11 (100 mg, 0.270 mmol) was dissolved in MeOH (1 mL). The solution was chilled in an ice bath, then was treated with HCl (2 M, 5 mL). After 15 mins, p-nitrobenzenediazonium tetrafluoroborate (67 mg, 0.283 mmol) was added to the solution in 3 portions over 15 mins, then stirred at 0° C. for an additional 1 h. The solution was then carefully neutralized with solid Na₂CO₃ until the pH value of the solution had risen above 7. The crude product was deposited onto a C₁₈ cartridge, washed with DI water, air-dried, followed by elution with MeOH. The solvent was evaporated under reduced pressure to give compound 12 (quantitative) and was used for the next step without further purification.

(E)-N¹-ethyl-N¹-(7-(ethylamino)-8-methyl-3H-phenoxazin-3-ylidene)-N³,N³,N³-trimethyl propane-1,3-diaminium (Example 2): Under N₂, compound 12 (50 mg, 0.096 mmol) and treated with p-toluenesulfonic acid monohydrate (55 mg, 0.289 mmol) were dissolved in ethanol (5 mL). The resulting solution was heated to 80° C., and stirred overnight. The solvent was evaporated under reduced pressure, and the residue was purified by reverse phase HPLC (MeCN/H₂O, 5-50%, linear gradient, TFA 0.1% as additive) to afford Example 2 (33.7 mg, 92%) as a dark blue solid.

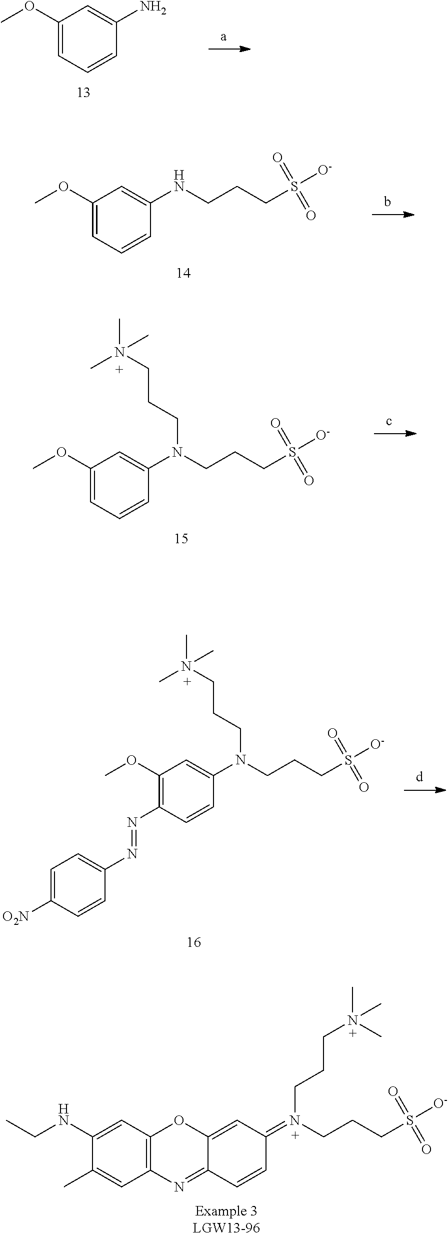

Scheme 3: Synthetic route to Example 3. Reagents and conditions: a) 1,3-Propanesultone, MeCN, 80° C.; (3-Bromopropyl)trimethylammonium bromide, Na₂CO₃, MeCN, 80° C.; c) I) 2M HCl, p-nitrobenzenediazonium tetrafluoroborate, 0° C.; II) Na₂CO₃, 0° C.; d) compound 21, HClO₄, 90% i-PrOH, 80° C.

3-((3-methoxyphenyl)amino)propane-1-sulfonate (14): To a suspension of compound 13 (2 g, 16.2 mmol) in anhydrous MeCN (20 mL) under N₂, was added 1,3-Propanesultone (2.02 g, 16.6 mmol) at rt. The reaction mixture was then heated up to 80° C., and stirred for 24 h. The solution was cooled down to rt, the solid was collected via vacuum filtration and washed with small portions of MeCN. The crude product was resuspended in EtOH (100 mL), and filtered through Celite. The filtrate was concentrated to dryness by a rotary evaporator to give compound 14 (3.74 g, 94%), and was used for the next step without further purification.

3-((3-methoxyphenyl)(3-(trimethylammonio)propyl)amino)propane-1-sulfonate (15): Compound 14 (2 g, 8.15 mmol), (3-Bromopropyl)trimethylammonium bromide (1.51 g, 8.32 mmol), and Na₂CO₃ (907 mg, 8.56 mmol) were suspended in anhydrous MeCN (50 mL) under N₂. The reaction mixture was then heated up to 80° C., and stirred for 24 h. The solution was cooled down to rt, the solid was collected via vacuum filtration and washed with small portions of MeCN. The crude product was resuspended in EtOH (100 mL), and filtered through Celite. The filtrate was concentrated to dryness by a rotary evaporator to give compound 15 (quantitative) and was used for the next step without further purification.

(E)-3-((3-methoxy-4-((4-nitrophenyl)diazenyl)phenyl)(3-(trimethylammonio)propyl)amino) propane-1-sulfonate (16): Compound 15 (500 mg, 1.36 mmol) was dissolved in MeOH (2 mL). The solution was chilled in an ice bath, then was treated with HCl (2 M, 20 mL). After 15 mins, p-nitrobenzenediazonium tetrafluoroborate (339 mg, 1.43 mmol) was added to the solution in 3 portions over 15 mins, then stirred at 0° C. for an additional 1 h. The solution was then carefully neutralized with solid Na₂CO₃ until the pH value of the solution had risen above 7. The crude product was deposited onto a C₁₈ cartridge, washed with DI water, air-dried, followed by elution with MeOH. The solvent was evaporated under reduced pressure to give compound 16 (quantitative) and was used for the next step without further purification.

(E)-3-((7-(ethylamino)-8-methyl-3H-phenoxazin-3-ylidene)(3-(trimethylammonio)propyl) ammonio)propane-1-sulfonate (Example 3): Compound 21 (50 mg, 0.304 mmol) was dissolved in a solution of i-PrOH/H₂O (9:1, 5 mL) at 80° C. for 30 min. Compound 16 (150 mg, 0.304 mmol) was added to the solution above in 3 portions over 15 mins. The reaction mixture was then treated with HCIO₄ (70%, 50 μL), and the resulting mixture was stirred for 12 h to give a dark-blue solution that was evaporated under reduced pressure, and the residue was purified by reverse phase HPLC (MeCN/H₂O, 5-50%, linear gradient, TFA 0.1% as additive) to afford the compound of Example No. 3 (71 mg, 49%) as a dark blue solid.

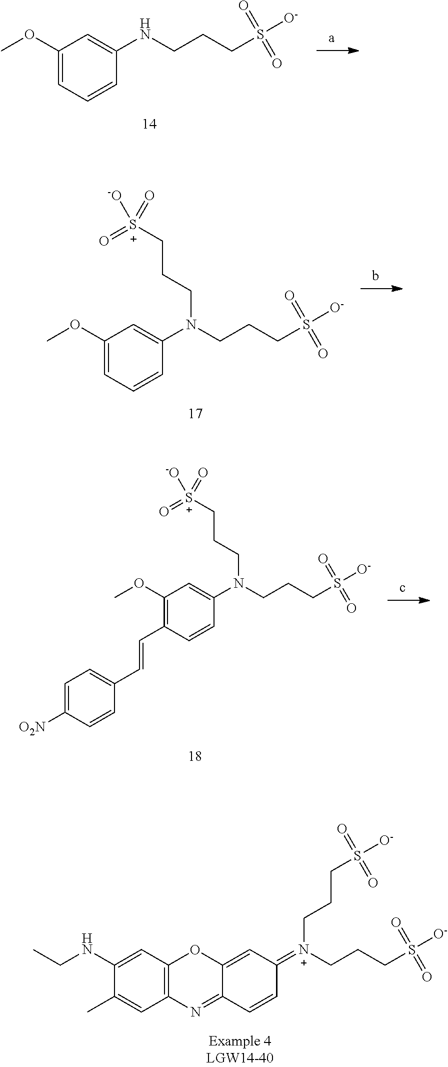

Example No. 4: 3,3′-((7-(ethylamino)-8-methyl-3H-phenoxazin-3-ylidene)ammonio)bis(propane-1-sulfonate) (LGW14-40) Scheme 4: Synthetic route to Example 4. Reagents and conditions: a) 1,3-Propanesultone, Na₂CO₃, MeCN, 80° C.; b) I) 2M HCl, p-nitrobenzenediazonium tetrafluoroborate, 0° C.; II) Na₂CO₃, 0° C.; c) compound 21, Trimethylsilylpolyphosphate, DMF, 80° C.

3,3′-((3-methoxyphenyl)azanediyl)bis(propane-1-sulfonate) (17): To a suspension of compound 14 (1 g, 4.08 mmol) and Na₂CO₃ (454 mg, 4.28 mmol) in anhydrous MeCN (20 mL) under N₂, was added 1,3-Propanesultone (508 mg, 4.16 mmol) at rt. The reaction mixture was then heated up to 80° C., and stirred for 24 h. The solution was cooled down to rt, the solid was collected via vacuum filtration and washed with small portions of MeCN. The crude product was resuspended in EtOH (100 mL), and filtered through celite. The filtrate was concentrated to dryness by a rotary evaporator, and the residue was purified by reverse phase HPLC (MeCN/H₂O, 5-50%, linear gradient, TFA 0.1% as additive) to afford compound 17 (1.39 g, 92%).

(E)-3,3′-((3-methoxy-4-((4-nitrophenyl)diazenyl)phenyl)azanediyl)bis(propane-1-sulfonate) (18): Compound 17 (610 mg, 1.66 mmol) was dissolved in MeOH (2 mL). The solution was chilled in an ice bath, then was treated with HCl (2 M, 20 mL). After 15 mins, p-nitrobenzenediazonium tetrafluoroborate (413 mg, 1.74 mmol) was added to the solution in 3 portions over 15 mins, then stirred at 0° C. for an additional 1 h. The solution was then carefully neutralized with solid Na₂CO₃ until the pH value of the solution had risen above 7. The crude product was deposited onto a C₁₈ cartridge, washed with DI water, air-dried, followed by elution with MeOH. The solvent was evaporated under reduced pressure to give compound 18 (quantitative), and was used for the next step without further purification.

3,3′-((7-(ethylamino)-8-methyl-3H-phenoxazin-3-ylidene)ammonio)bis(propane-1-sulfonate) (Example 4): Compound 21 (5 mg, 0.0304 mmol) and 18 (15.7 mg, 0.0304 mmol) were dissolved DMF (0.5 mL), to which Trimethylsilylpolyphosphate (10 μL) was added. The resulting solution was heated at 80° C. overnight. The crude product was then directly purified by reverse phase HPLC (MeCN/H₂O, 5-50%, linear gradient, TFA 0.1% as additive) to afford the compound of Example 4.

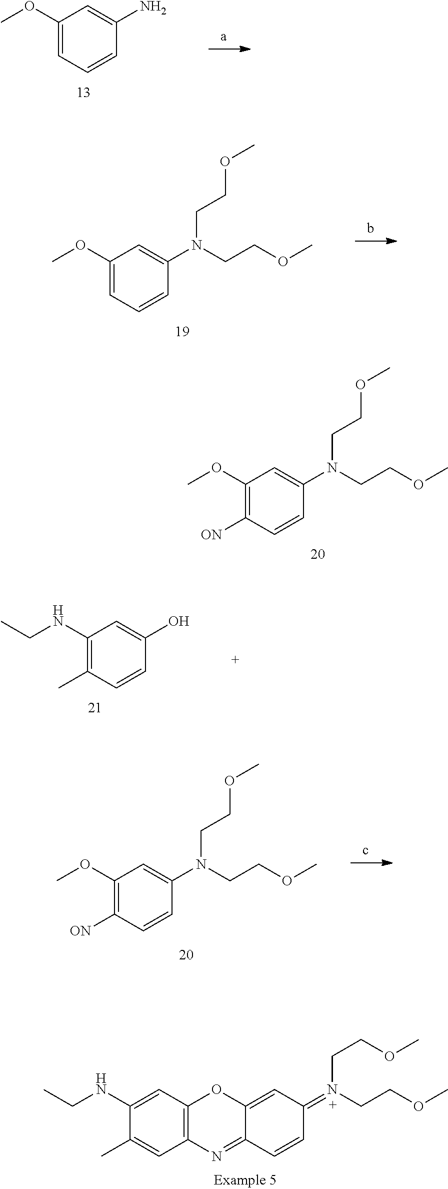

Example No. 5: N-(7-(ethylamino)-8-methyl-3H-phenoxazin-3-ylidene)-2-methoxy-N-(2-methoxyethyl)ethan-1-aminium (LGW06-45) Scheme 5: Synthetic route to Example 5. Reagents and conditions: a) 2-bromoethylmethylther, K₂CO₃, MeCN, 80° C.; b) 2 M HCl, NaNO₂, 0° C.; ii) K₂CO₃, 0° C.; c) HCIO₄, 90% i-PrOH, 80° C.

3-methoxy-N,N-bis(2-methoxyethyl)aniline (19): Compound 13 (1 g, 8.12 mmol), 2-bromoethylmethylther (3.42 g, 24.36 mmol), and K₂CO₃ (2.24 g, 16.2 mmol) were suspended in anhydrous MeCN (20 mL) under N₂. The reaction mixture was then heated to 80° C. and stirred for 24 h before diluted with DCM (50 mL). The solid was removed via vacuum filtration through Celite. The solvent was removed using a rotary evaporator and the residue was purified by flash column chromatography with silica gel (25 g), using DCM/Hexane as eluent to give compound 19 (1.62 g, 83%) as clear oil.

3-methoxy-N,N-bis(2-methoxyethyl)-4-nitrosoaniline (20): Compound 19 (1.2 g, 5.01 mmol) was dissolved in an ice-cold 2 M HCl solution (15 mL). To this solution was added NaNO₂ (381 mg, 5.52 mmol) portion-wise over 1 h while the temperature of the solution was maintained below 5° C., such that no brown NOx vapors were observed. The reaction mixture was stirred for an additional 2 h. The solution was carefully basified with solid K₂CO₃ until the pH value of the solution had risen above 8. The resulting precipitate was filtered through a Buchner funnel and washed with small portions of ice-cold DI water. The title compound was obtained (1.03 g, 77%) as a green solid, which was used for the next step without further purification.

N-(7-(ethylamino)-8-methyl-3H-phenoxazin-3-ylidene)-2-methoxy-N-(2-methoxyethyl)ethan-1-aminium (Example 5): Compound 21 (50 mg, 0.304 mmol) was dissolved in a solution of i-PrOH/H₂O (9:1, 5 mL) at 80° C. for 30 min. Compound 20 (86 mg, 0.319 mmol) was added to the solution above in 3 portions over 15 mins. The reaction mixture was then treated with HCIO₄ (70%, 50 μL), and the resulting mixture was stirred for 12 h to give a dark-blue solution that was evaporated under reduced pressure. The residue was purified by flash column chromatography with silica gel (25 g), using a mobile phase of CHCl₃ and MeOH containing 0.5% formic acid to give compound of Example 5 (62 mg, 55%).

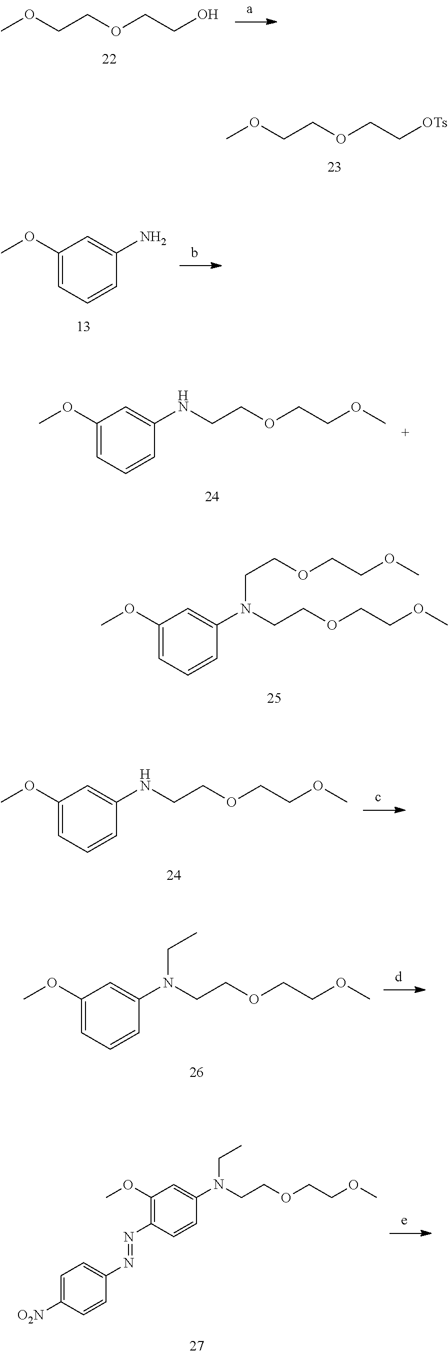

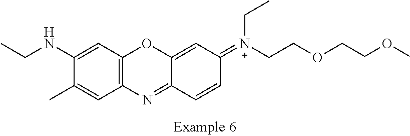 [SMILES: CCNc1cc2oc3c/c(=[N+](\CC)CCOCCOC)ccc-3nc2cc1C]

Example No. 6 (LGW14-12): Scheme 6: Synthetic route to Example 6. Reagents and conditions: a) TsCl, NaOH, THF/H₂O, 0° C. to rt; b) compound 23, K₂CO₃, MeCN, 80° C.; c) Etl, Na₂CO₃, MeCN, 80° C.; d) I) 2M HCl, p-nitrobenzenediazonium tetrafluoroborate, 0° C.; II) K₂CO₃, 0° C.; e) compound 21, HClO₄, 90% i-PrOH, 80° C.

2-(2-methoxyethoxy)ethyl 4-methylbenzenesulfonate (23): To a THF solution (25 mL) of diethylene glycol methyl ether (5 g, 41.6 mmol) was added NaOH (20%, 25 mL). The resulting solution was chilled in an ice bath before TsCl (9.52 g, 49.9 mmol) in THF (25 mL) was added dropwise. The reaction mixture was stirred at 0° C. for 2 h, and warmed up to rt overnight. The reaction mixture was poured into HCl (5%) solution. The product was extracted with extracted with CHCl₃ (4×50 mL). The combined organic layers were washed with brine and dried over anhydrous Na₂SO₄, then concentrated in vacuo. The title compound was obtained (quantitative) and was used for the next step without further purification.

3-methoxy-N-(2-(2-methoxyethoxy)ethyl)aniline (24) and 3-methoxy-N,N-bis(2-(2-methoxy ethoxy)ethyl)aniline (25): Compound 13 (1 g, 8.12 mmol), compound 23 (4.46 g, 16.2 mmol), and K₂CO₃ (2.24 g, 16.2 0.798 mmol) were suspended in anhydrous MeCN (20 mL) under N₂. The reaction mixture was then heated to 80° C. and stirred for 24 h before diluted with DCM (50 mL). The solid was removed via vacuum filtration through Celite. The solvent was removed using a rotary evaporator and the residue was purified by flash column chromatography with silica gel (25 g), using DCM/Hexane as eluent to give compound 24 (0.618 g, 34%) and 25 (1.06 g, 40

N-ethyl-3-methoxy-N-(2-(2-methoxyethoxy)ethyl)aniline (26): Compound 24 (500 mg, 2.22 mmol), Etl (363 mg, 2.33 mmol), and Na₂CO₃ (353 mg, 3.33 mmol) were suspended in anhydrous MeCN (20 mL) under N₂. The reaction mixture was then heated to 80° C. and stirred for 24 h before diluted with DCM (50 mL). The solid was removed via vacuum filtration through Celite. The solvent was removed using a rotary evaporator and the residue was purified by flash column chromatography with silica gel (25 g), using DCM/Hexane as eluent to give compound 26 (449 g, 80%) as a clear oil.

(E)-N-ethyl-3-methoxy-N-(2-(2-(2-methoxyethoxy)ethoxy)ethyl)-4-((4-nitrophenyl)diazenyl) aniline (27): Compound 26 (300 mg, 1.18 mmol) was dissolved in MeOH (2 mL). The solution was chilled in an ice bath, then was treated with HCl (2 M, 20 mL). After 15 mins, p-nitrobenzenediazonium tetrafluoroborate (295 mg, 1.24 mmol) was added to the solution in 3 portions over 15 mins, then stirred at 0° C. for an additional 1 h. The solution was then carefully neutralized with solid Na₂CO₃ until the pH value of the solution had risen above 7, and exacted with DCM (3×50 mL). The combined organic layers were washed with brine and dried over anhydrous Na₂SO₄, then concentrated in vacuo. The title compound was obtained (quantitative) and was used for the next step without further purification. V

(E)-N-ethyl-N-(7-(ethylamino)-8-methyl-3H-phenoxazin-3-ylidene)-2-(2-methoxyethoxy)ethan-1-aminium (Example 6): Compound 21 (50 mg, 0.304 mmol) was dissolved in a solution of i-PrOH/H₂O (9:1, 5 mL) at 80° C. for 30 min. Compound 27 (122 mg, 0.304 mmol) was added to the solution above in 3 portions over 15 mins. The reaction mixture was then treated with HClO₄ (70%, 50 μL), and the resulting mixture was stirred for 12 h to give a dark-blue solution that was evaporated under reduced pressure. The residue was purified by flash column chromatography with silica gel (25 g), using a mobile phase of CHCl₃ and MeOH to give the compound of Example 6 (69 mg, 59%).

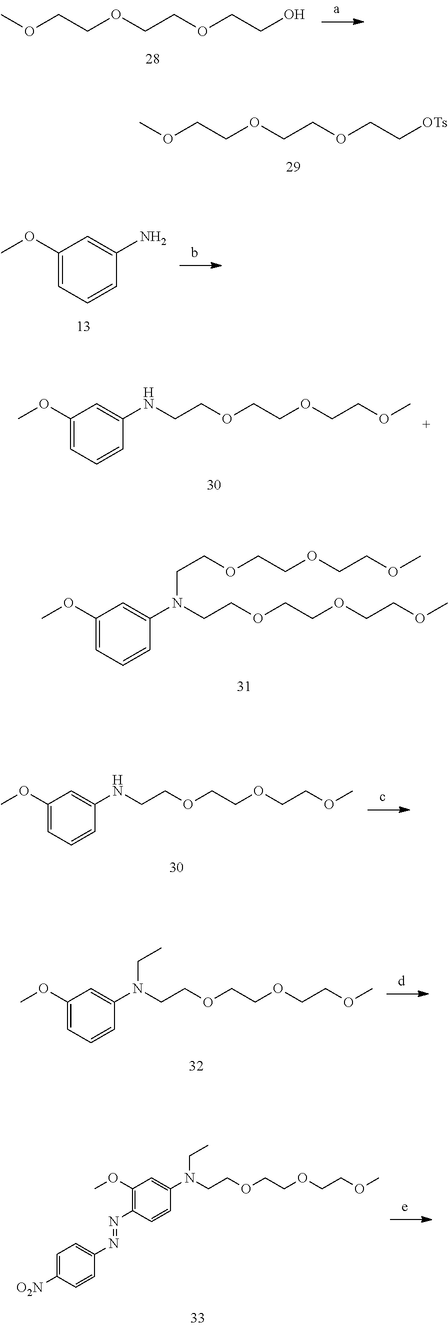

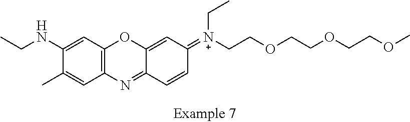 [SMILES: CCNc1cc2oc3c/c(=[N+](\CC)CCOCCOCCOC)ccc-3nc2cc1C]

Example No. 7: (E)-N-ethyl-N-(7-(ethylamino)-8-methyl-3H-phenoxazin-3-ylidene)-2-(2-(2-methoxyethoxy)ethoxy)ethan-1-aminium (LGW13-77) Scheme 7: Synthetic route to Example No. 7. Reagents and conditions: a) TsCl, NaOH, THF/H₂O, 0° C. to rt; b) compound 29, K₂CO₃, MeCN, 80° C.; c) Etl, Na₂CO₃, MeCN, 80° C.; d) I) 2M HCl, p-nitrobenzenediazonium tetrafluoroborate, 0° C.; II) K₂CO₃, 0° C.; e) compound 21, HClO₄, 90% i-PrOH, 80° C.

2-(2-(2-methoxyethoxy)ethoxy)ethyl 4-methylbenzenesulfonate (29): To a THF solution (25 mL) of Triethylene glycol monomethyl ether (5 g, 30.5 mmol) was added NaOH (20%, 25 mL). The resulting solution was chilled in an ice bath before TsCl (6.97 g, 36.5 mmol) in THF (25 mL) was added dropwise. The reaction mixture was stirred at 0° C. for 2 h, and warmed up to rt overnight. The reaction mixture was poured into HCl (5%) solution. The product was extracted with extracted with CHCl₃ (4×50 mL). The combined organic layers were washed with brine and dried over anhydrous Na₂SO₄, then concentrated in vacuo. The title compound was obtained (quantitative) and was used for the next step without further purification.

3-methoxy-N-(2-(2-(2-methoxyethoxy)ethoxy)ethyl)aniline (30) and 3-methoxy-N,N-bis(2-(2-(2-methoxyethoxy)ethoxy)ethyl)aniline (31): Compound 13 (500 mg, 4.06 mmol), compound 29 (5.17 g, 16.2 mmol), and K₂CO₃ (2.24 g, 16.2 mmol) were suspended in anhydrous MeCN (10 mL) under N₂. The reaction mixture was then heated to 80° C. and stirred for 24 h before diluted with DCM (25 mL). The solid was removed via vacuum filtration through Celite. The solvent was removed using a rotary evaporator and the residue was purified by flash column chromatography with silica gel (25 g), using DCM/Hexane as eluent to give compound 30 (458 g, 42%) and 31 (0.601 g, 36%).

N-ethyl-3-methoxy-N-(2-(2-(2-methoxyethoxy)ethoxy)ethyl)aniline (32): Compound 30 (400 mg, 1.49 mmol), Etl (236 mg, 1.51 mmol), and Na₂CO₃ (165 mg, 1.56 mmol) were suspended in anhydrous MeCN (10 mL) under N₂. The reaction mixture was then heated to 80° C. and stirred for 24 h before diluted with DCM (25 mL). The solid was removed via vacuum filtration through Celite. The solvent was removed using a rotary evaporator and the residue was purified by flash column chromatography with silica gel (25 g), using DCM/Hexane as eluent to give compound 32 (376 mg, 85%).

(E)-N-ethyl-3-methoxy-N-(2-(2-(2-methoxyethoxy)ethoxy)ethyl)-4-((4-nitrophenyl)diazenyl) aniline (33): Compound 32 (300 mg, 1.01 mmol) was dissolved in MeOH (2 mL). The solution was chilled in an ice bath, then was treated with HCl (2 M, 20 mL). After 15 mins, p-nitrobenzenediazonium tetrafluoroborate (251 mg, 1.06 mmol) was added to the solution in 3 portions over 15 mins, then stirred at 0° C. for an additional 1 h. The solution was then carefully neutralized with solid Na₂CO₃ until the pH value of the solution had risen above 7, and exacted with DCM (3×50 mL). The combined organic layers were washed with brine and dried over anhydrous Na₂SO₄, then concentrated in vacuo. The title compound was obtained (quantitative) and was used for the next step without further purification.

(E)-N-ethyl-N-(7-(ethylamino)-8-methyl-3H-phenoxazin-3-ylidene)-2-(2-(2-methoxyethoxy) ethoxy)ethan-1-aminium (Example 7): Compound 21 (50 mg, 0.304 mmol) was dissolved in a solution of i-PrOH/H₂O (9:1, 5 mL) at 80° C. for 30 min. Compound 33 (136 mg, 0.304 mmol) was added to the solution above in 3 portions over 15 mins. The reaction mixture was then treated with HClO₄ (70%, 50 μL), and the resulting mixture was stirred for 12 h to give a dark-blue solution that was evaporated under reduced pressure. The residue was purified by flash column chromatography with silica gel (25 g), using a mobile phase of CHCl₃ and MeOH to give the compound of Example 7 (53 mg, 41%).

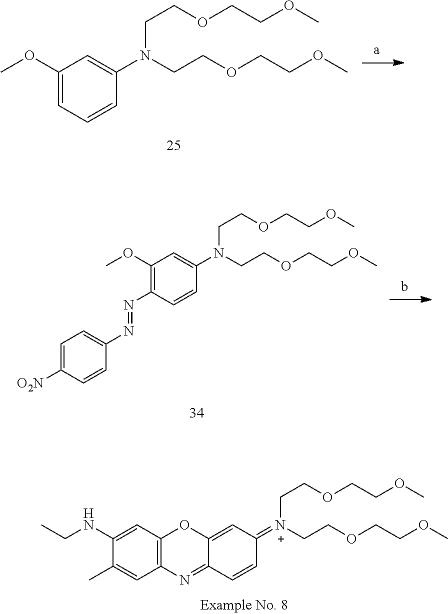

Example No. 8: N-(7-(ethylamino)-8-methyl-3H-phenoxazin-3-ylidene)-2-(2-methoxyethoxy)-N-(2-(2-methoxyethoxy)ethyl)ethan-1-aminium (LGW13-98) Scheme 8: Synthetic route to Example 8. Reagents and conditions: a) I) 2M HCl, p-nitrobenzenediazonium tetrafluoroborate, 0° C.; II) K₂CO₃, 0° C.; b) compound 21, HClO₄, 90% i-PrOH, 80° C.

(E)-3-methoxy-N,N-bis(2-(2-methoxyethoxy)ethyl)-4-((4-nitrophenyl)diazenyl)aniline (34): Compound 25 (246 mg, 0.751 mmol) was dissolved in MeOH (1 mL). The solution was chilled in an ice bath, then was treated with HCl (2 M, 10 mL). After 15 mins, p-nitrobenzenediazonium tetrafluoroborate (187 mg, 0.789 mmol) was added to the solution in 3 portions over 15 mins, then stirred at 0° C. for an additional 1 h. The solution was then carefully neutralized with solid Na₂CO₃ until the pH value of the solution had risen above 7, and exacted with DCM (3×50 mL). The combined organic layers were washed with brine and dried over anhydrous Na₂SO₄, then concentrated in vacuo. The title compound was obtained (quantitative) and was used for the next step without further purification.

N-(7-(ethylamino)-8-methyl-3H-phenoxazin-3-ylidene)-2-(2-methoxyethoxy)-N-(2-(2-methoxy ethoxy)ethyl)ethan-1-aminium (Example 8): Compound 21 (50 mg, 0.304 mmol) was dissolved in a solution of i-PrOH/H₂O (9:1, 5 mL) at 80° C. for 30 min. Compound 34 (145 mg, 0.304 mmol) was added to the solution above in 3 portions over 15 mins. The reaction mixture was then treated with HCIO₄ (70%, 50 μL), and the resulting mixture was stirred for 12 h to give a dark-blue solution that was evaporated under reduced pressure. The residue was purified by flash column chromatography with silica gel (25 g), using a mobile phase of CHCl₃ and MeOH to give the compound of Example 8 (54 mg, 39%).

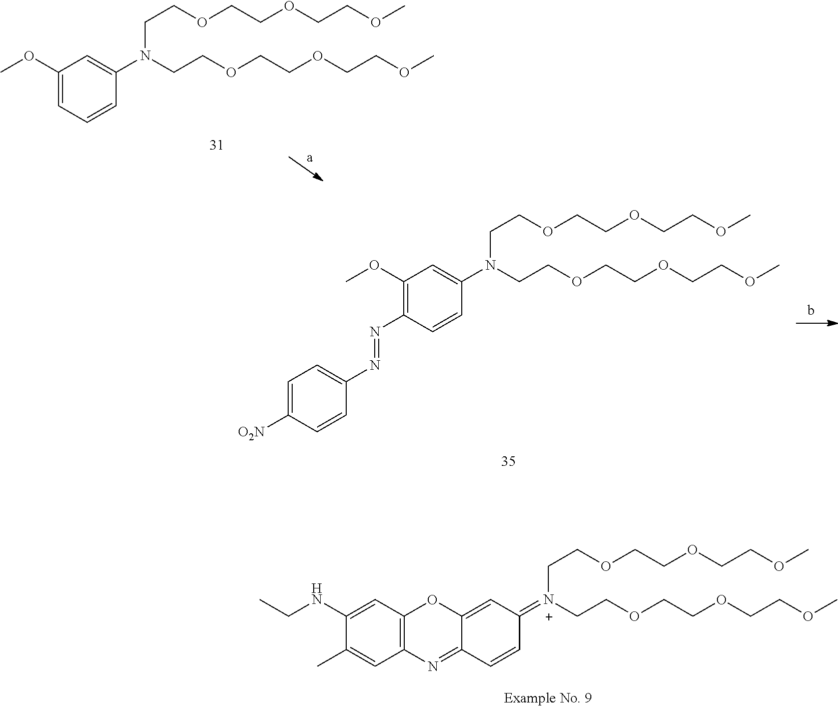

### Example No. 9: N-(7-(ethylamino)-8-methyl-3H-phenoxazin-3-ylidene)-2-(2-(2-methoxyethoxy)ethoxy)-N-(2-(2-(2-methoxyethoxy)ethoxy)ethyl)ethan-1-aminium

(LGW13-34) Scheme 9: Synthetic route to Example 9. Reagents and conditions: a) I) 2M HCl, p-nitrobenzenediazonium tetrafluoroborate, 0° C.; II) K₂CO₃, 0° C.; b) compound 21, HCIO₄, 90% i-PrOH, 80° C.

(E)-3-methoxy-N,N-bis(2-(2-(2-methoxyethoxy)ethoxy)ethyl)-4-((4-nitrophenyl)diazenyl)aniline (35): Compound 31 (300 mg, 0.722 mmol) was dissolved in MeOH (1 mL). The solution was chilled in an ice bath, then was treated with HCl (2 M, 10 mL). After 15 mins, p-nitrobenzenediazonium tetrafluoroborate (180 mg, 0.758 mmol) was added to the solution in 3 portions over 15 mins, then stirred at 0° C. for an additional 1 h. The solution was then carefully neutralized with solid Na₂CO₃ until the pH value of the solution had risen above 7, and exacted with DCM (3×50 mL). The combined organic layers were washed with brine and dried over anhydrous Na₂SO₄, then concentrated in vacuo. The title compound was obtained (quantitative) and was used for the next step without further purification.

N-(7-(ethylamino)-8-methyl-3H-phenoxazin-3-ylidene)-2-(2-(2-methoxyethoxy)ethoxy)-N-(2-(2-(2-methoxyethoxy)ethoxy)ethyl)ethan-1-aminium (Example 9): Compound 21 (30 mg, 0.183 mmol) was dissolved in a solution of i-PrOH/H₂O (9:1, 4 mL) at 80° C. for 30 min. Compound 35 (103 mg, 0.183 mmol) was added to the solution above in 3 portions over 15 mins. The reaction mixture was then treated with HCIO₄ (70%, 40 μL), and the resulting mixture was stirred for 12 h to give a dark-blue solution that was evaporated under reduced pressure. The residue was purified by flash column chromatography with silica gel (25 g), using a mobile phase of CHCl₃ and MeOH to give the compound of Example 9 (27 mg, 27%).

### Example No. 10: (E)-N-ethyl-8-methyl-3-(2,5,11,14,17-pentaoxa-8λ⁴-azaoctadecan-8-ylidene)-3H-phenoxazin-7-amine

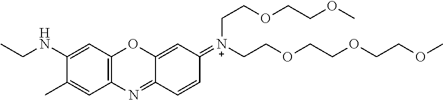 [SMILES: CCNc1cc2oc3c/c(=[N+](\CCOCCOC)CCOCCOCCOC)ccc-3nc2cc1C]

In addition to tosylate compounds 23 (synthesis of Example No. 6) and 29 (synthesis of Example No. 7), above, it is understood that compounds of the present disclosure may be made with the use of additional tosylate compounds known in the art. Illustrative and non-limiting examples include:
2-methoxyethyl 4-methylbenzenesulfonate (CAS Reg. No. 17178-10-8);
2-ethoxyethyl 4-methylbenzenesulfonate (CAS Reg. No. 17178-11-9);
2-(vinyloxy)ethyl 4-methylbenzenesulfonate (CAS Reg. No. 99051-18-0);
2-propoxyethyl 4-methylbenzenesulfonate (CAS Reg. No. 52497-47-9);
2-isopropoxyethyl 4-methylbenzenesulfonate (CAS Reg. No. 51218-98-5);
2-(allyloxy)ethyl 4-methylbenzenesulfonate (CAS Reg. No. 50563-72-9);
2-isobutoxyethyl 4-methylbenzenesulfonate (CAS Reg. No. 1852889-86-1);
2-(tert-butoxy)ethyl 4-methylbenzenesulfonate (CAS Reg. No. 108366-80-9);
2-((2-methylallyl)oxy)ethyl 4-methylbenzenesulfonate (CAS Reg. No. 64011-00-3);
2-(but-3-en-2-yloxy)ethyl 4-methylbenzenesulfonate (CAS Reg. No. 1628446-55-8);
2-(isopentyloxy)ethyl 4-methylbenzenesulfonate (CAS Reg. No. 915184-71-3);
2-(but-3-yn-1-yloxy)ethyl 4-methylbenzenesulfonate (CAS Reg. No. 1418561-91-7);
2-(2-methoxyethoxy)ethyl 4-methylbenzenesulfonate (CAS Reg. No. 50586-80-6);
2-(2-ethoxyethoxy)ethyl 4-methylbenzenesulfonate (CAS Reg. No. 54176-27-1);
2-(2-(vinyloxy)ethoxy)ethyl 4-methylbenzenesulfonate (CAS Reg. No. 117731-86-9);
2-(2-propoxyethoxy)ethyl 4-methylbenzenesulfonate (CAS Reg. No. 1709852-20-9);
2-(2-(allyloxy)ethoxy)ethyl 4-methylbenzenesulfonate (CAS Reg. No. 84183-96-0);
2-(2-(pentyloxy)ethoxy)ethyl 4-methylbenzenesulfonate (CAS Reg. No. 50964-16-4);
2-(2-(prop-2-yn-1-yloxy)ethoxy)ethyl 4-methylbenzenesulfonate (CASE Reg. No. 1119249-30-7);
2-(2-(tert-butoxy)ethoxy)ethyl 4-methylbenzenesulfonate (CAS Reg. No. 1431853-87-0);
2-(2-(isopentyloxy)ethoxy)ethyl 4-methylbenzenesulfonate (CAS Reg. No. 1359296-24-4);
2-(2-(pentyloxy)ethoxy)ethyl 4-methylbenzenesulfonate (CAS Reg. No. 2248492-16-0);
2-(2-(hexyloxy)ethoxy)ethyl 4-methylbenzenesulfonate (CAS Reg. No. 187748-60-3);
2-(2-(2-methoxyethoxy)ethoxy)ethyl 4-methylbenzenesulfonate (CAS Reg. No. 62921-74-8);
2-(2-(2-ethoxyethoxy)ethoxy)ethyl 4-methylbenzenesulfonate (CAS Reg. No. 62921-75-9);
2-(2-(2-propoxyethoxy)ethoxy)ethyl 4-methylbenzenesulfonate (CAS Reg. No. 64820-20-8);
2-(2-(2-(allyloxy)ethoxy)ethoxy)ethyl 4-methylbenzenesulfonate (CAS Reg. No. 84183-97-1);
2-(2-(2-(prop-2-yn-1-yloxy)ethoxy)ethoxy)ethyl 4-methylbenzenesulfonate (CAS Reg. No. 888009-94-7);
11-ethoxy-3,6,9,12-tetraoxatetradecyl 4-methylbenzenesulfonate (CAS Reg. No. 881920-24-7);
14-ethoxy-3,6,9,12,15-pentaoxaheptadecyl 4-methylbenzenesulfonate (CAS Reg. No. 1630091-41-6);
2,5,9,12-tetraoxatridecan-7-yl 4-methylbenzenesulfonate (CAS Reg. No. 1644403-15-5);
2,5,8,12,15,18-hexaoxanonadecan-10-yl 4-methylbenzenesulfonate (CAS Reg. No. 1644403-20-2);
3,6,9,13,16,19-hexaoxahenicosan-11-yl 4-methylbenzenesulfonate (CAS Reg. No. 508224-19-9);
2,5,8,11,15,18,21,24-octaoxapentacosan-13-yl 4-methylbenzenesulfonate (CAS Reg. No. 214851-23-7);
2,5,8,11,14,18,21,24,27,30-decaoxahentriacontan-16-yl 4-methylbenzenesulfonate (CAS Reg. No. 2346589-70-4);
2-(2-((tert-butyldimethylsilyl)oxy)ethoxy)ethyl 4-methylbenzenesulfonate (CAS Reg. No. 131326-40-4); and
2,2-dimethyl-3,6,9-trioxa-2-silaundecan-11-yl 4-methylbenzenesulfonate (CAS Reg. No. 472968-83-5).
2,2,3,3-tetramethyl-4,7,10-trioxa-3-siladodecan-12-yl 4-methylbenzenesulfonate (CAS Reg. No. 199484-66-7).

TABLE 1
Current Small Molecule Organic Fluorophores with Nerve Specificity & Potential
for a Near Infrared Fluorophore Upon Derivatization.

| Nerve-Specific | Excitation | Emission | # Nerve-Specific |
|---|---|---|---|
| Fluorophore | (nm) | (nm) | probes |
| Nerve-specific peptide | 492, 646 | 517, 662 | 2 |
| Stilebene derivatives | 350, 363 | 415, 419 | |
| Coumarin fluorophore | 407 | 551 | 1 |
| Distyrylbenzene (DSB) derivatives | 247-396 | 431-656 | 242 |
| Styryl pyridinium (FM) derivatives | 490-519 | 628-815 | 8 |
| Oxazine fluorophore | 616, 625 | 635, 650 | 2 |
| Tricarbocyanine (TCC) fluorophore | 760 | 800 | 1 |
| Nerve-Specific | | | Potential for NIR |
| Fluorophore | Advantages | Disadvantages | nerve-specificity |
| Nerve-specific | Peripheral nerve | Too large for BNB | Low—conjugation to |
| peptide | specificity | penetration, nonspecific | NIR fluorophores |
| | | skin & adipose signal | possible, but nerve SBR |
| | | | low due to nonspecific |
| | | | tissue accumulation |
| Stilebene | Reported myelin | UV excitation & | Low—too few double |
| derivatives | specificity in brain | emission, nerve- | bonds to reach NIR |
| | | specificity unknown | excitation or emission |
| Coumarin | Reported myelin | Fluorescence emission | Low—too few double |
| fluorophore | specificity in brain | solvent dependent, | bonds to reach NIR |
| | | nerve-specificity | excitation or emission |
| | | unknown | |
| Distyrylbenzene | Highlights all | Fluorescence emission | Medium—SAR for |
| (DSB) derivatives | nerves in CNS & | solvent dependent, no | nerve-specificity known, |
| | PNS when | current NIR derivatives | sufficient double bonds |
| | administered | | to reach NIR emission, |
| | systemically | | NIR excitation |
| | | | challenging |
| Styryl pyridinium | NIR emissions + | Visible excitation, limited | Medium—Direct |
| (FM) derivatives | some nerve | nerve specificity following | administration may |
| | specificity | systemic administration | provide nerve-specificity, |
| | | (DRG & TG only) | NIR excitation may be |
| | | | synthetically available |
| Oxazine | Highlights all | Excitation & emission not | High—Current excitation |
| fluorophore | nerves in CNS & | yet in the NIR region | & emission close to NIR |
| | PNS when | (650-900 nm) | with strong possibility for |
| | administered | | synthetic tuning, highly |
| | systemically | | nerve-specific |
| Tricarbocyanine | NIR excitation & | No nerve partitioning | Medium—NIR excitation |
| (TCC) | emission, reported | following systemic | & emission, may be |
| fluorophore | myelin specificity | administration, no nerve | synthetically tunable to |
| | in the brain | accumulation after 4 hrs | create improved nerve- |
| | | + rapid clearance | specificity |

### Solubility Testing

Each compound was dissolved at high concentration (50 mg/mL) in dimethyl sulfoxide (DMSO) to create a stock solution. Stock solutions were spiked into ultrapure water beginning with a 50 μg/mL concentration and the aqueous solution was vortexed to mix. Resulting solutions were centrifuged at 15,000 rpm for 2 min and examined for a pellet or precipitation signifying insoluble compound. If no pellet or precipitation was observed, compound concentrations were increased in increments of 50 μg/mL, mixed, and re-centrifuged until a pellet or precipitation was observed. Water solubility was recorded as the highest concentration at which no insoluble compound was observed with an upper cutoff in water solubility measurement at 5 mg/mL as DMSO concentrations increased to >10%.

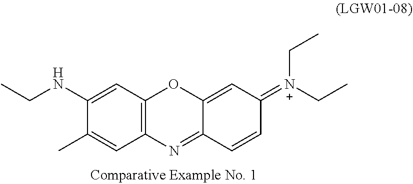 [SMILES: CCNc1cc2oc3cc(=[N+](CC)CC)ccc-3nc2cc1C]

### 3-(diethyl-λ4-azaneylidene)-N-ethyl-8-methyl-3H-phenoxazin-7-amine (LGW01-08)

| | NOAEL (mg/kg) | Water Solubility (mg/ml) |
|---|---|---|
| Comparative | 3 | 0.1 |
| Example No. 1 | | |
| Example No. 1 | 30 | >5 |
| Example No. 2 | 2 | >5 |
| Example No. 5 | 4 | 3 |
| Example No. 7 | 5 | 4 |
| Example No. 8 | 10 | >5 |
| Example No. 9 | 30 | >5 |

NOAEL = no-observed-adverse-effect-level

### Absorption and Fluorescence Spectroscopy.

All screening candidates were solubilized in phosphate buffered saline (PBS), pH 7.4 at a concentration of 20 μM. Absorbance spectra were collected using a SpectraMax M5 spectrometer with a Microplate reader (Molecular Devices, Sunnyvale, Calif.). All absorbance spectra were reference corrected. Extinction coefficients for all oxazine derivatives were calculated using Beer's Law plots of absorbance versus concentration. Fluorescence emission spectra were collected with excitation at 590 nm. Relative quantum yields were calculated using the oxazine 1 as reference standard.¹⁷ All data reported were the average of multiple measurements.

### Water Solubility Measurements.

Each screening candidate was dissolved in a 1 mL mixture of chloroform and methanol (equal volume) with final stock concentrations ranging from 10 to 50 mM. The solvent was then removed in vacuo before 200 μL of DI water was added. The test sample was then vortexed before sonicated in an ultrasonic bath for 30 minutes. The undissolved pellet was removed by centrifugation at 13,000 rpm for 5 minutes. The supernatant was sampled and diluted with water before measured for absorbance using a SpectraMax M5 spectrometer with a Microplate reader (Molecular Devices, Sunnyvale, Calif.). The water solubility of each screening candidate was then calculated using Beer's Law plots of absorbance versus concentration. The water solubility concentration unit (mM) of each sample was then converted and reported as mg/mL.

### Experimental Log D Measurements.

Each screening candidate was dissolved in DMSO at a concentration of 10 mM. The stock solution was sampled (2 μL) and added to a 1 mL mixture of 1-octanol and PBS buffer (equal volume). The solution was then vortexed for 30 mins at room temperature before centrifuged at 13,000 rpm for 5 minutes. The PBS buffer and 1-octanol layers were separated and measured for absorbance using a SpectraMax M5 spectrometer with a Microplate reader (Molecular Devices, Sunnyvale, Calif.). Sample concentration in each phase was then calculated using Beer's Law plots of absorbance versus concentration. The experimental Log D value for each screening candidates was calculated using the equation below.

$$LogD = Log ( \frac{Sample concentration in PBS buffer}{S ample concetration in 1 - o c t a n o l} )$$

### Nerve-Specificity Screening Using Direct/Topical Administration.

Each compound was screened for its tissue-specificity using a previously published direct/topical administration strategy in murine brachial plexus and sciatic nerves.¹⁶ Each compound from the Oxazine library was formulated in phosphate buffered saline (PBS), pH 7.4, at 125 μM. 100 μL of the formulated candidates were incubated on the exposed brachial plexus or sciatic nerve for 5 minutes. The fluorophore containing solution was removed and the area was irrigated with saline 18 times to remove any unbound fluorophore. Co-registered fluorescence and color images were collected of each stained area 30 minutes after Oxazine direct/topical administration using a custom-built macroscopic imaging system with 620/60 nm excitation and 700/75 nm bandpass emission filters. Custom written MatLab code was used to analyze the tissue specific fluorescence where regions of interest were selected on the nerve, muscle and adipose tissue using the white light images. These regions of interest were then analyzed on the co-registered matched fluorescence images permitting assessment of the nerve to muscle and nerve to adipose ratios.

### Nerve-Specificity Screening Using Systemic Administration

Each compound was screened for its tissue-specificity using a previously published systemic administration strategy in murine brachial plexus and sciatic nerves.¹⁶ Each compound from the compound library was formulated in PBS at a concentration of 2-10 mM. The formulated fluorophore was administered intravenously before exposing the brachial plexus and sciatic nerves. Co-registered fluorescence and color images were collected of each nerve site using a custom-built macroscopic imaging system with 620/60 nm excitation and 700/75 nm bandpass emission filters. Custom written MatLab code was used to analyze the tissue specific fluorescence where regions of interest were selected on the nerve, muscle and adipose tissue using the white light images. These regions of interest were then analyzed on the co-registered matched fluorescence images permitting assessment of the nerve-to-muscle and nerve-to-adipose ratios in blinded manner.

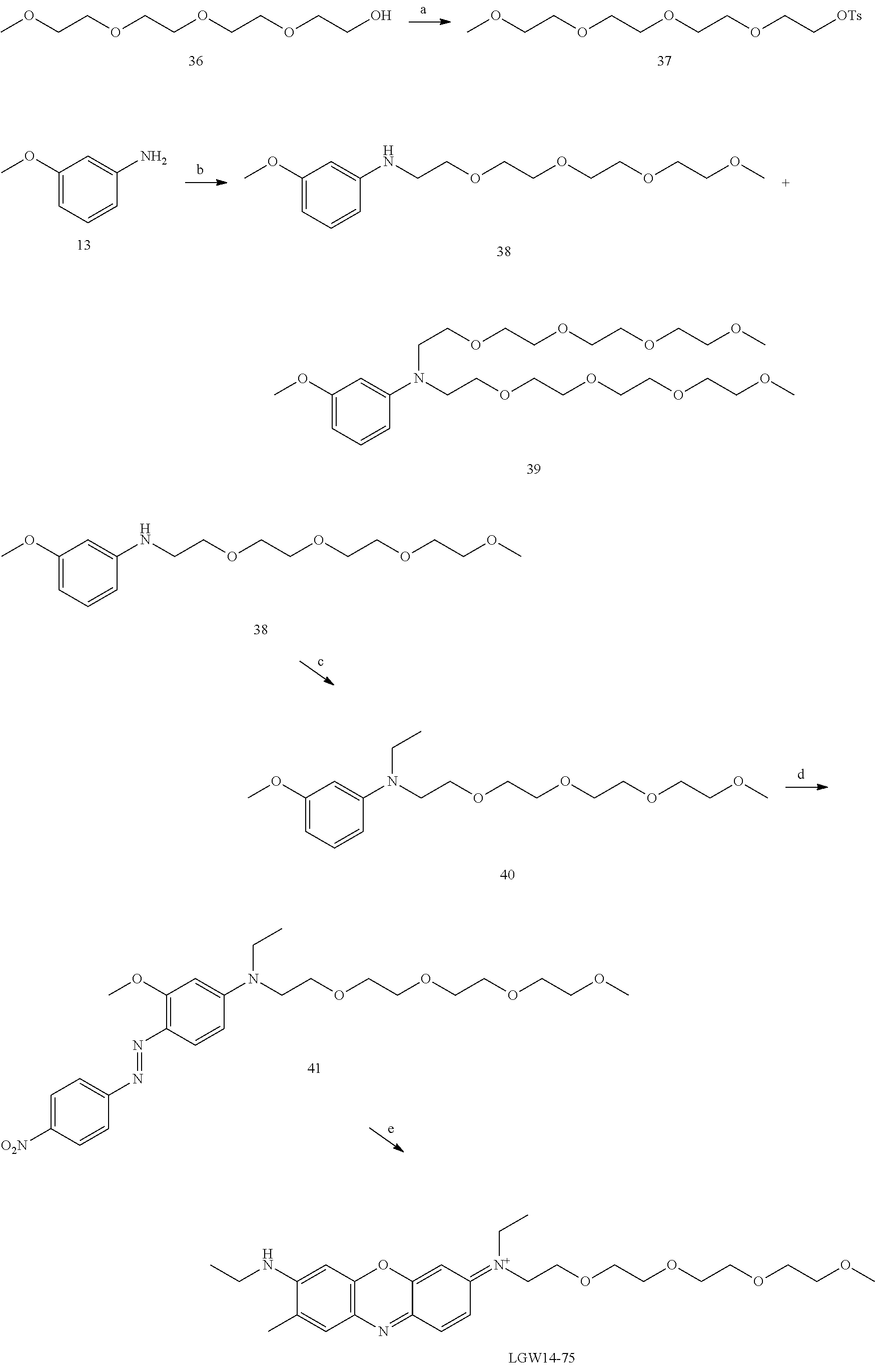

Scheme 10: Synthetic route to LGW14-75. Reagents and conditions: a) TsCl, NaOH, THF/H₂O, 0° C. to rt; b) compound 37, K₂CO₃, MeCN, 80° C.; c) Etl, Na₂CO₃, MeCN, 80° C.; d) I) 2M HCl, p-nitrobenzenediazonium tetrafluoroborate, 0° C.; II) K₂CO₃, 0° C.; e) compound 21, HCIO₄, 90% i-PrOH, 80° C.

2,5,8,11-tetraoxatridecan-13-yl 4-methylbenzenesulfonate (37): To a THF solution (25 mL) of Tetraethyleneglycol monomethyl ether 36 (5 g, 24.0 mmol) was added NaOH (20%, 25 mL). The resulting solution was chilled in an ice bath before TsCl (6.01 g, 28.8 mmol) in THF (25 mL) was added dropwise. The reaction mixture was stirred at 0° C. for 2 h, and warmed up to rt overnight. The reaction mixture was poured into HCl (5%) solution. The product was extracted with extracted with CHCl₃ (4×50 mL). The combined organic layers were washed with brine and dried over anhydrous Na₂SO₄, then concentrated in vacuo. The title compound was obtained (quantitative) and was used for the next step without further purification.

N-(3-methoxyphenyl)-2,5,8,11-tetraoxatridecan-13-amine (38) and N-(3-methoxyphenyl)-N-(2,5,8,11-tetraoxatridecan-13-yl)-2,5,8,11-tetraoxatridecan-13-amine (39): Compound 13 (1.0 g, 8.12 mmol), compound 37 (5.89 g, 16.2 mmol), and K₂CO₃ (2.24 g, 16.2 mmol) were suspended in anhydrous MeCN (10 mL) under N₂. The reaction mixture was then heated to 80° C. and stirred for 48 h before diluted with DCM (25 mL). The solid was removed via vacuum filtration through Celite. The solvent was removed using a rotary evaporator and the residue was purified by flash column chromatography with silica gel, using EtOAc/Hexane as eluent to give compound 38 (1.92 mg, 75%) and 39 (827 mg, 20%).

N-ethyl-N-(3-methoxyphenyl)-2,5,8,11-tetraoxatridecan-13-amine (40): Compound 38 (500 mg, 1.60 mmol), Etl (261 mg, 1.68 mmol), and Na₂CO₃ (254 mg, 2.39 mmol) were suspended in anhydrous MeCN (10 mL) under N₂. The reaction mixture was then heated to 80° C. and stirred for 24 h before diluted with DCM (25 mL). The solid was removed via vacuum filtration through Celite. The solvent was removed using a rotary evaporator and the residue was purified by flash column chromatography with silica gel, using DCM/Hexane as eluent to give compound 40 (395 mg, 73%) as clear oil.

(E)-N-ethyl-N-(3-methoxy-4-((4-nitrophenyl)diazenyl)phenyl)-2,5,8,11-tetraoxatridecan-13-amine (41): Compound 40 (500 mg, 1.46 mmol) was dissolved in MeOH (2 mL). The solution was chilled in an ice bath, then was treated with HCl (2 M, 20 mL). After 15 mins, p-nitrobenzenediazonium tetrafluoroborate (364 mg, 1.54 mmol) was added to the solution in 3 portions over 15 mins, then stirred at 0° C. for an additional 2 h. The solution was then carefully neutralized with solid Na₂CO₃ until the pH value of the solution had risen above 7, and exacted with DCM (3×50 mL). The combined organic layers were washed with brine and dried over anhydrous Na₂SO₄, then concentrated in vacuo. The title compound was obtained (quantitative) and was used for the next step without further purification.

Example No. 10—(E)-N-ethyl-N-(7-(ethylamino)-8-methyl-3H-phenoxazin-3-ylidene)-2,5,8,11-tetraoxatridecan-13-aminium (LGW14-75): Compound 21 (50 mg, 0.304 mmol) was dissolved in a solution of i-PrOH/H₂O (9:1, 5 mL) at 80° C. for 10 min. Compound 41 (149 mg, 0.304 mmol) was added to the solution above followed by addition of HCIO₄ (70%, 50 μL), and the resulting mixture was stirred at 80° C. for an additional 12 h to give a dark-blue solution that was evaporated under reduced pressure. The residue was purified by flash column chromatography with silica gel using a mobile phase of CHCl₃ and MeOH containing 0.5% formic acid to give compound LGW14-75 (19.4 mg, 13.5%).

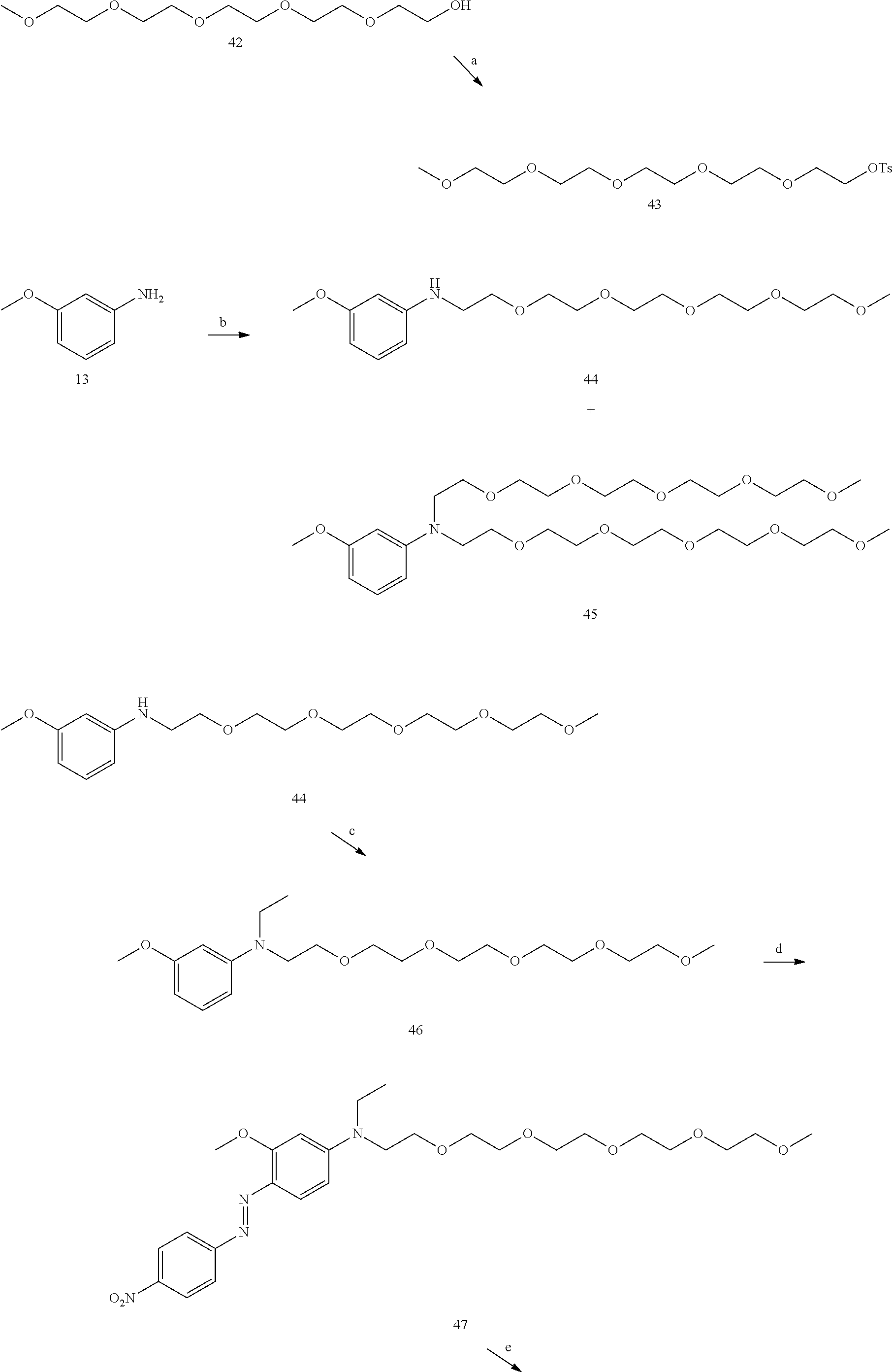

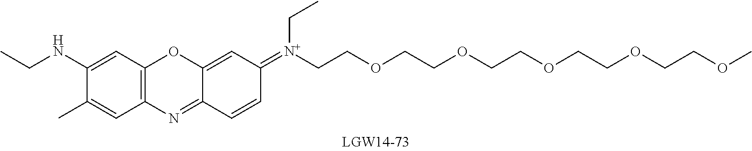 [SMILES: CCNc1cc2oc3c/c(=[N+](\CC)CCOCCOCCOCCOCCOC)ccc-3nc2cc1C]

Scheme 11: Synthetic route to LGW14-73. Reagents and conditions: a) TsCl, NaOH, THF/H₂O, 0° C. to rt; b) compound 43, K₂CO₃, MeCN, 80° C.; c) Etl, Na₂CO₃, MeCN, 80° C.; d) I) 2M HCl, p-nitrobenzenediazonium tetrafluoroborate, 0° C.; II) K₂CO₃, 0° C.; e) compound 21, HClO₄, 90% i-PrOH, 80° C.

2,5,8,11,14-pentaoxahexadecan-16-yl 4-methylbenzenesulfonate (43): To a THF solution (25 mL) of pentaethylene glycol monomethyl ether 42 (5.0 g, 19.8 mmol) was added NaOH (20%, 25 mL). The resulting solution was chilled in an ice bath before TsCl (4.96 g, 23.8 mmol) in THF (25 mL) was added dropwise. The reaction mixture was stirred at 0° C. for 2 h, and warmed up to rt overnight. The reaction mixture was poured into HCl (5%) solution. The product was extracted with extracted with CHCl₃ (4×50 mL). The combined organic layers were washed with brine and dried over anhydrous Na₂SO₄, then concentrated in vacuo. The title compound was obtained (quantitative) and was used for the next step without further purification.

N-(3-methoxyphenyl)-2,5,8,11,14-pentaoxahexadecan-16-amine (44) and N-(2,5,8,11,14-pentaoxahexadecan-16-yl)-N-(3-methoxyphenyl)-2,5,8,11,14-pentaoxahexadecan-16-amine (45): Compound 13 (1.0 g, 8.12 mmol), compound 43 (6.60 g, 16.2 mmol), and K₂CO₃ (2.24 g, 16.2 mmol) were suspended in anhydrous MeCN (10 mL) under N₂. The reaction mixture was then heated to 80° C. and stirred for 48 h before diluted with DCM (25 mL). The solid was removed via vacuum filtration through Celite. The solvent was removed using a rotary evaporator and the residue was purified by flash column chromatography with silica gel, using EtOAc/Hexane as eluent to give compound 44 (1.46 g, 50%) and 45 (814 mg, 17%).

N-ethyl-N-(3-methoxyphenyl)-2,5,8,11,14-pentaoxahexadecan-16-amine (46): Compound 44 (1.0 g, 2.80 mmol), Etl (458 mg, 2.94 mmol), and Na₂CO₃ (445 mg, 4.20 mmol) were suspended in anhydrous MeCN (20 mL) under N₂. The reaction mixture was then heated to 80° C. and stirred for 24 h before diluted with DCM (40 mL). The solid was removed via vacuum filtration through Celite. The solvent was removed using a rotary evaporator and the residue was purified by flash column chromatography with silica gel, using DCM/Hexane as eluent to give compound 46 (779 mg, 72%).

(E)-N-ethyl-N-(3-methoxy-4-((4-nitrophenyl)diazenyl)phenyl)-2,5,8,11,14-pentaoxahexadecan-16-amine (47): Compound 46 (500 mg, 1.30 mmol) was dissolved in MeOH (2 mL). The solution was chilled in an ice bath, then was treated with HCl (2 M, 20 mL). After 15 mins, p-nitrobenzenediazonium tetrafluoroborate (323 mg, 1.36 mmol) was added to the solution in 3 portions over 15 mins, then stirred at 0° C. for an additional 2 h. The solution was then carefully neutralized with solid Na₂CO₃ until the pH value of the solution had risen above 7, and exacted with DCM (3×50 mL). The combined organic layers were washed with brine and dried over anhydrous Na₂SO₄, then concentrated in vacuo. The title compound was obtained (quantitative) and was used for the next step without further purification.

Example No. 11—(E)-N-ethyl-N-(7-(ethylamino)-8-methyl-3H-phenoxazin-3-ylidene)-2,5,8,11,14-pentaoxahexadecan-16-aminium (LGW14-75): Compound 21 (50 mg, 0.304 mmol) was dissolved in a solution of i-PrOH/H₂O (9:1, 5 mL) at 80° C. for 10 min. Compound 47 (163 mg, 0.304 mmol) was added to the solution above followed by addition of HCIO₄ (70%, 50 μL), and the resulting mixture was stirred at 80° C. for an additional 12 h to give a dark-blue solution that was evaporated under reduced pressure. The residue was purified by flash column chromatography with silica gel using a mobile phase of CHCl₃ and MeOH containing 0.5% formic acid to give compound LGW14-73 (19.1 mg, 12%).

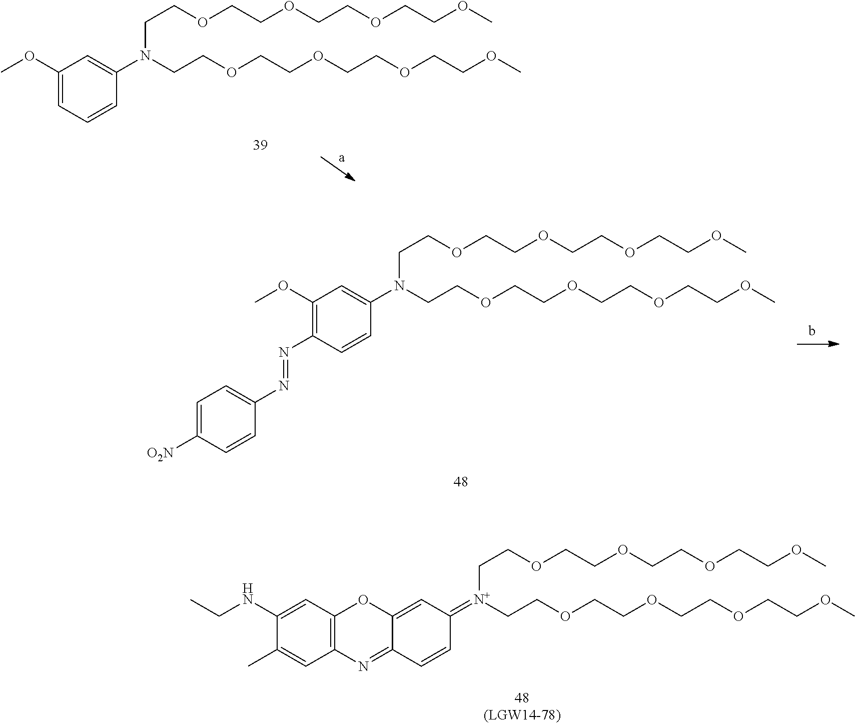

Scheme 12: Synthetic route to LGW14-78. Reagents and conditions: a) I) 2M HCl, p-nitrobenzenediazonium tetrafluoroborate, 0° C.; II) K₂CO₃, 0° C.; b) compound 21, HCIO₄, 90% i-PrOH, 80° C.

(E)-N-(3-methoxy-4-((4-nitrophenyl)diazenyl)phenyl)-N-(2,5,8,11-tetraoxatridecan-13-yl)-2,5,8,11-tetraoxatridecan-13-amine (48): Compound 39 (500 mg, 0.993 mmol) was dissolved in MeOH (2 mL). The solution was chilled in an ice bath, then was treated with HCl (2 M, 20 mL). After 15 mins, p-nitrobenzenediazonium tetrafluoroborate (247 mg, 1.04 mmol) was added to the solution in 3 portions over 15 mins, then stirred at 0° C. for an additional 2 h. The solution was then carefully neutralized with solid Na₂CO₃ until the pH value of the solution had risen above 7, and exacted with DCM (3×50 mL). The combined organic layers were washed with brine and dried over anhydrous Na₂SO₄, then concentrated in vacuo. The title compound was obtained (quantitative) and was used for the next step without further purification.

Example No. 12—N-(7-(ethylamino)-8-methyl-3H-phenoxazin-3-ylidene)-N-(2,5,8,11-tetraoxatridecan-13-yl)-2,5,8,11-tetraoxatridecan-13-aminium (LGW14-78): Compound 21 (50 mg, 0.304 mmol) was dissolved in a solution of i-PrOH/H₂O (9:1, 5 mL) at 80° C. for 10 min. Compound 48 (199 mg, 0.304 mmol) was added to the solution above followed by addition of HCIO₄ (70%, 50 μL), and the resulting mixture was stirred at 80° C. for an additional 12 h to give a dark-blue solution that was evaporated under reduced pressure. The residue was purified by flash column chromatography with silica gel using a mobile phase of CHCl₃ and MeOH containing 0.5% formic acid to give compound LGW14-78 (28.2 mg, 15%).

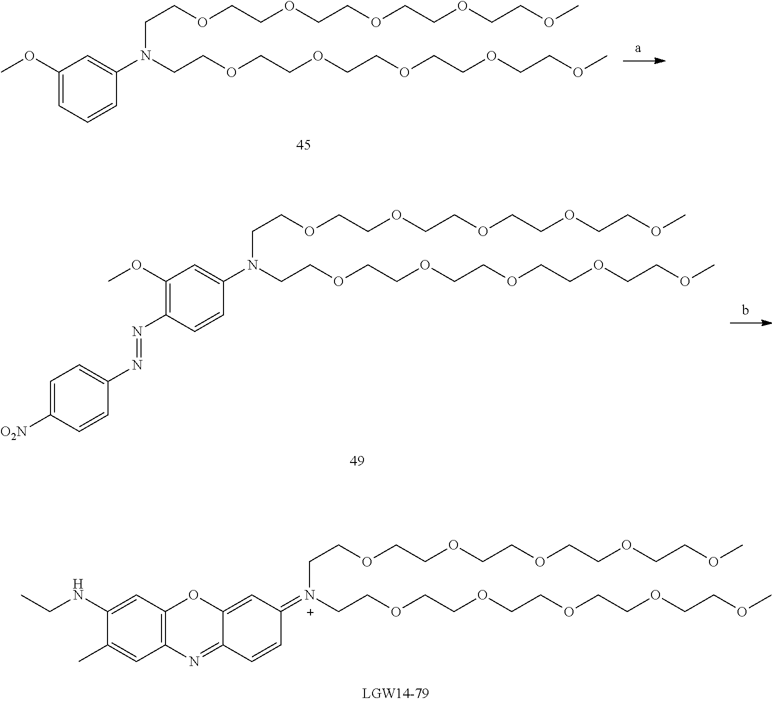

Scheme 13: Synthetic route to LGW14-79. Reagents and conditions: a) I) 2M HCl, p-nitrobenzenediazonium tetrafluoroborate, 0° C.; II) K₂CO₃, 0° C.; b) compound 21, HCIO₄, 90% i-PrOH, 80° C.

(E)-N-(2,5,8,11,14-pentaoxahexadecan-16-yl)-N-(3-methoxy-4-((4-nitrophenyl)diazenyl)phenyl)-2,5,8,11,14-pentaoxahexadecan-16-amine (49): Compound 45 (500 mg, 0.845 mmol) was dissolved in MeOH (2 mL). The solution was chilled in an ice bath, then was treated with HCl (2 M, 20 mL). After 15 mins, p-nitrobenzenediazonium tetrafluoroborate (210 mg, 0.887 mmol) was added to the solution in 3 portions over 15 mins, then stirred at 0° C. for an additional 2 h. The solution was then carefully neutralized with solid Na₂CO₃ until the pH value of the solution had risen above 7, and exacted with DCM (3×50 mL). The combined organic layers were washed with brine and dried over anhydrous Na₂SO₄, then concentrated in vacuo. The title compound was obtained (quantitative) and was used for the next step without further purification.

Example No. 15—N-(7-(ethylamino)-8-methyl-3H-phenoxazin-3-ylidene)-N-(2,5,8,11,14-pentaoxahexadecan-16-yl)-2,5,8,11,14-pentaoxahexadecan-16-aminium (LGW14-79): Compound 21 (50 mg, 0.304 mmol) was dissolved in a solution of i-PrOH/H₂O (9:1, 5 mL) at 80° C. for 10 min. Compound 49 (225 mg, 0.304 mmol) was added to the solution above followed by addition of HCIO₄ (70%, 50 μL), and the resulting mixture was stirred at 80° C. for an additional 12 h to give a dark-blue solution that was evaporated under reduced pressure. The residue was purified by flash column chromatography with silica gel using a mobile phase of CHCl₃ and MeOH containing 0.5% formic acid to give compound LGW14-79 (7.2 mg, 3.3%).

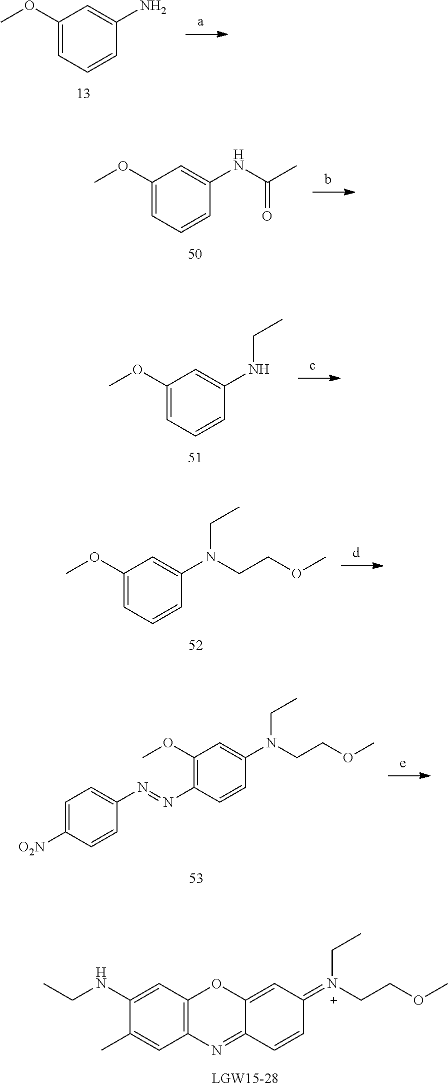

Scheme 14: Synthetic route to LGW15-28. Reagents and conditions: a) Ac₂O, H₂O, 50° C. to rt; b) BH₃-THF, THF, 0° C. to rt; c) 2-Bromoethyl methyl ether, MeCN, 80° C.; d) I) 2M HCl, p-nitrobenzenediazonium tetrafluoroborate, 0° C.; II) K₂CO₃, 0° C.; e) compound 21, HCIO₄, 90% i-PrOH, 80° C.

N-(3-methoxyphenyl)acetamide (50): Compound 13 (2 g, 16.2 mmol) was suspended in 50 mL DI water, to which acetic anhydride (4.61 mL, 48.7 mmol) was added slowly. The reaction mixture was placed in an ultrasonication bath for 1 min, then was stirred in a water bath at 50° C. for 10 min. The resulting solution was stirred overnight at rt. The reaction mixture was chilled in an ice bath and carefully neutralized with NaOH (10%) aqueous solution. The aqueous solution was then extracted with DCM (3×50 mL), and the combined organic layers were washed with brine, dried over anhydrous Na₂SO₄, then concentrated in vacuo. The product was left in the funnel and air dried overnight to afford compound 50 (2.22 g, 83%) as a light brown oil, which was solidified upon cooling in the fridge, compound 50 was used for the next step without further purification.

N-ethyl-3-methoxyaniline (51): A solution of 50 (2.0 g, 12.2 mmol) in anhydrous THF (35 mL) was stirred in an ice bath under N₂ for 30 mins. Borane tetrahydrofuran complex solution (1 M, 35 mL) was added to the solution above using a syringe pump over 30 mins, while maintaining the temperature of the solution below 5° C. The resulting reaction mixture was left in the ice bath and slowly warmed to rt. After 24 h, the solution was placed in an ice bath again, and excess borane reagent was destroyed by carefully adding MeOH until no gas evolved. The solvent was evaporated under reduced pressure, and the residue was purified by flash column chromatography with silica gel, using DCM/Hexane as eluent to obtain compound 51 (1.39 g, 75%) as a solid.

N-ethyl-3-methoxy-N-(2-methoxyethyl)aniline (52): Compound 51 (1.0 g, 6.61 mmol), 2-Bromoethyl methyl ether (1.39 g, 9.92 mmol), and K₂CO₃ (1.37 g, 9.92 mmol) were suspended in anhydrous MeCN (20 mL) under N₂. The reaction mixture was then heated to 80° C. and stirred for 24 h before a second addition of 2-Bromoethyl methyl ether (1.39 g, 9.92 mmol). The reaction mixture was stirred for another 48 h before diluted with DCM (40 mL). The solid was removed via vacuum filtration through Celite. The solvent was removed using a rotary evaporator and the residue was purified by flash column chromatography with silica gel, using DCM/Hexane as eluent to give compound 52 (359 mg, 26%).

(E)-N-ethyl-3-methoxy-N-(2-methoxyethyl)-4-((4-nitrophenyl)diazenyl)aniline (53): Compound 52 (300 mg, 1.43 mmol) was dissolved in MeOH (2 mL). The solution was chilled in an ice bath, then was treated with HCl (2 M, 20 mL). After 15 mins, p-nitrobenzenediazonium tetrafluoroborate (374 mg, 1.58 mmol) was added to the solution in 3 portions over 15 mins, then stirred at 0° C. for an additional 2 h. The solution was then carefully neutralized with solid Na₂CO₃ until the pH value of the solution had risen above 7, and exacted with DCM (3×50 mL). The combined organic layers were washed with brine and dried over anhydrous Na₂SO₄, then concentrated in vacuo. The title compound was obtained (quantitative) and was used for the next step without further purification.

Example No. 16—(E)-N-ethyl-N-(7-(ethylamino)-8-methyl-3H-phenoxazin-3-ylidene)-2-methoxyethan-1-aminium (LGW15-28): Compound 21 (142 mg, 0.864 mmol) was dissolved in a solution of i-PrOH/H₂O (9:1, 5 mL) at 80° C. for 10 min. Compound 53 (295 mg, 0.823 mmol) was added to the solution above followed by addition of HCIO₄ (70%, 50 μL), and the resulting mixture was stirred at 80° C. for an additional 4 h to give a dark-blue solution that was evaporated under reduced pressure. The residue was purified by flash column chromatography with silica gel using a mobile phase of CHCl₃ and MeOH containing 0.5% formic acid to give compound LGW15-28 (58.1 mg, 21%).

HPLC-MS characterization of oxazine derivative library. HPLC-MS was used to quantify the purity of each oxazine derivative via area under the curve (AUC) analysis of the absorbance at 254 nm (left) and mass to charge (m/z) ratio in positive ion mode (right). Sample (5 μL) was injected into a C₁₈ column (Poroshell 120, 2.1×50 mm, 2.7 micron), and eluted with a solvent system of A (H₂O, 0.1% formic acid) and B (Acetonitrile, 0.1% formic acid) at 0.4 mL/min, from A/B=95/5 to 5/95 over 6 min, maintained at A/B=5/95 for additional 2 min. Ions were detected in positive ion mode by setting the capillary voltage at 4 kV and gas temperature at 350° C.

HPLC-MS and purity analysis of oxazine derivative library.

| | | | | Mass | |
|---|---|---|---|---|---|
| | Retention Time | Measured | Calculated | Accuracy | |
| ID | (min) | m/z | m/z | (ppm) | Purity |
| LGW01-08 | 5.25 | 310.1903 | 310.1914 | −3.55 | 98% |
| LGW06-45 | 5.04 | 370.2127 | 370.2125 | 0.54 | 97% |
| LGW11-63 | 4.58 | 404.1660 | 404.1639 | 5.20 | >99% |
| LGW12-60 | 4.86 | 191.1366 | 191.1361 | 2.62 | 98% |
| LGW13-34 | 4.96 | 546.3197 | 546.3174 | 4.21 | 98% |
| LGW13-77 | 5.11 | 428.2542 | 428.2544 | −0.47 | 98% |
| LGW13-96 | 3.75 | 475.2372 | 475.2374 | −0.42 | >99% |
| LGW13-98 | 5.01 | 458.2659 | 458.2649 | 2.18 | 98% |
| LGW14-12 | 5.11 | 384.2294 | 384.2282 | 3.12 | 97% |
| LG W14-40 | 3.89 | 498.1374 | 498.1364 | 2.01 | >99% |
| LGW14-73 | 5.12 | 516.3087 | 516.3068 | 3.68 | >99% |
| LGW14-75 | 5.11 | 472.2809 | 472.2806 | 0.64 | >99% |
| LGW14-78 | 5.02 | 634.3691 | 634.3698 | −1.10 | 96% |
| LGW14-79 | 5.10 | 722.4241 | 722.4222 | 2.63 | >99% |
| LGW15-28 | 5.16 | 340.2026 | 340.2020 | 1.76 | 97% |

### REFERENCES

1. Chance, B. Near-infrared images using continuous, phase-modulated, and pulsed light with quantitation of blood and blood oxygenation. Annals of the New York Academy of Sciences 838, 29-45 (1998).
2. Vahrmeijer, A. L., Hutteman, M., van der Vorst, J. R., van de Velde, C. J. & Frangioni, J. V. Image-guided cancer surgery using near-infrared fluorescence. Nature reviews. Clinical oncology 10, 507-518 (2013).
3. Frangioni, J. V. In vivo near-infrared fluorescence imaging. Current opinion in chemical biology 7, 626-634 (2003).
4. Gibbs, S. L. Near infrared fluorescence for image-guided surgery. Quantitative imaging in medicine and surgery 2, 177-187 (2012).
5. Whitney, M. A. et al. Fluorescent peptides highlight peripheral nerves during surgery in mice. Nature biotechnology 29, 352-356 (2011).
6. Wu, C. et al. Molecular probes for imaging myelinated white matter in CNS. Journal of medicinal chemistry 51, 6682-6688 (2008).
7. Wang, C. et al. In situ fluorescence imaging of myelination. The journal of histochemistry and cytochemistry: official journal of the Histochemistry Society 58, 611-621 (2010).
8. Stankoff, B. et al. Imaging of CNS myelin by positron-emission tomography. Proceedings of the National Academy of Sciences of the United States of America 103, 9304-9309 (2006).
9. Cotero, V. E. et al. Intraoperative fluorescence imaging of peripheral and central nerves through a myelin-selective contrast agent. Molecular imaging and biology: MIB: the official publication of the Academy of Molecular Imaging 14, 708-717 (2012).
10. Gibbs, S. L. et al. Structure-activity relationship of nerve-highlighting fluorophores. PloS one 8, e73493 (2013).
11. Gibbs-Strauss, S. L. et al. Nerve-highlighting fluorescent contrast agents for image-guided surgery. Molecular imaging 10, 91-101 (2011).
12. Meyers, J. R. et al. Lighting up the senses: FM1-43 loading of sensory cells through nonselective ion channels. The Journal of neuroscience: the official journal of the Society for Neuroscience 23, 4054-4065 (2003).
13. Gibbs-Strauss, S. L. et al. Molecular imaging agents specific for the annulus fibrosus of the intervertebral disk. Molecular imaging 9, 128-140 (2010).
14. Park, M. H. et al. Prototype nerve-specific near-infrared fluorophores. Theranostics 4, 823-833 (2014).
15. Wang, C. et al. Longitudinal near-infrared imaging of myelination. The Journal of neuroscience: the official journal of the Society for Neuroscience 31, 2382-2390 (2011).
16. Barth, C. W. & Gibbs, S. L. Direct Administration of Nerve-Specific Contrast to Improve Nerve Sparing Radical Prostatectomy. Theranostics 7, 573-593 (2017).
17. Rurack, K. & Spieles, M. Fluorescence Quantum Yields of a Series of Red and Near Infrared Dyes Emitting at 600-1000 nm. Analytical Chemistry 83, 1232-1242 (2011).

What is claimed:

1. A compound of Formula (I): 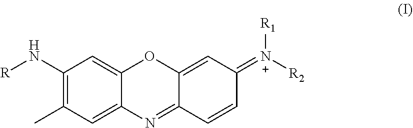
wherein:
R is a straight or branched alkyl chain of from 2 to 12 carbon atoms;
R₁ is selected from the group of methyl, ethyl, n-propyl, isopropyl, —(CH₂)ₙ₁—SO₃⁻, —(CH₂)ₙ₁—N⁺(CH₃)₃, —CH₂—CH₂—O—X₁, —CH₂—CH₂—O—[CH₂—CH₂—O]ₙ₂—X₁, —CH₂—CH₂—CH₂—O—X₁, and —CH₂—CH₂—CH₂—O—[CH₂—CH₂—CH₂—O]ₙ₃—X₁; 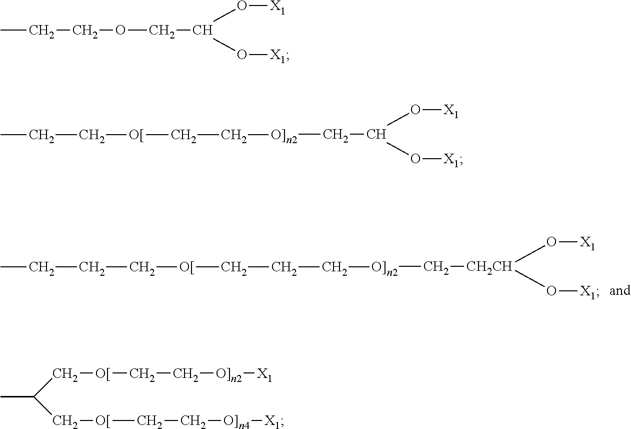
R₂ is selected from the group of —(CH₂)ₙ₁—SO₃⁻, —(CH₂)ₙ₁—N⁺(CH₃)₃, —CH₂—CH₂—O—X₁, —CH₂—CH₂—O—[CH₂—CH₂—O]ₙ₄—X₁, —CH₂—CH₂—CH₂—O—X₁, and —CH₂—CH₂—CH₂—O—[CH₂—CH₂—CH₂—O]ₙ₅—X₁; 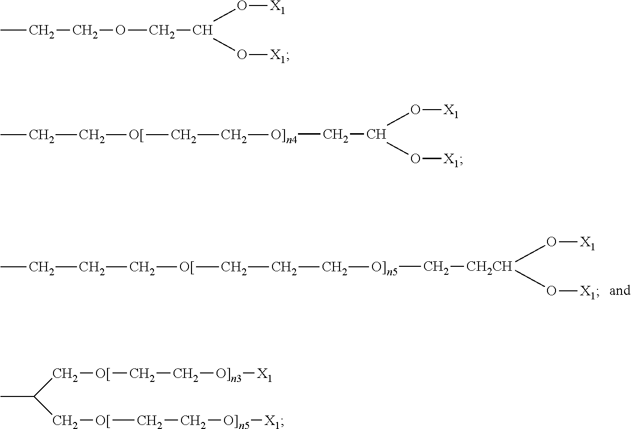
X₁ in each instance is independently selected from C₁-C₆ straight or branched alkyl, C₂-C₆ straight or branched alkenyl, C₁-C₆ straight or branched alkynyl, and —Si(C₁-C₄ alkyl)₃;
n1 is an integer independently selected in each instance from the group of 1, 2, 3, and 4;
n2 is an integer independently selected in each instance from the group of 1, 2, 3, 4, 5, 6, 7, 8, 9, and 10;
n3 is an integer independently selected in each instance from the group of 1, 2, 3, 4, 5, 6, 7, 8, 9, and 10;
n4 is an integer independently selected in each instance from the group of 1, 2, 3, 4, 5, 6, 7, 8, 9, and 10; and
n5 is an integer independently selected in each instance from the group of 1, 2, 3, 4, 5, 6, 7, 8, 9, and 10;
with the proviso that the sum of n2+n4 is not greater than 10;
with the proviso that the sum of n3+n5 is not greater than 10;
with the proviso that the sum of n2+n5 is not greater than 10; and
with the proviso that the sum of n3+n4 is not greater than 10.

2. The compound of claim 1, having the Formula (II): 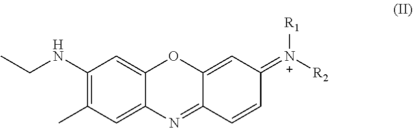
wherein:
R₁ is selected from the group of methyl, ethyl, n-propyl, isopropyl, —(CH₂)ₙ₁—SO₃⁻, —(CH₂)ₙ₁—N⁺(CH₃)₃;
—CH₂—CH₂—O—X₁;
—CH₂—CH₂—O—[CH₂—CH₂—O]ₙ₂—X₁;
—CH₂—CH₂—CH₂—O—X₁; and
—CH₂—CH₂—CH₂—O—[CH₂—CH₂—CH₂—O]ₙ₃—X₁;
R₂ is selected from the group of —(CH₂)ₙ₁—SO₃⁻, —(CH₂)ₙ₁—N⁺(CH₃)₃;
—CH₂—CH₂—O—X₁;
—CH₂—CH₂—O—[CH₂—CH₂—O]ₙ₄—X₁;
—CH₂—CH₂—CH₂—O—X₁; and
—CH₂—CH₂—CH₂—O—[CH₂—CH₂—CH₂—O]ₙ₅—X₁;
X₁ in each instance is independently selected from C₁-C₆ straight or branched alkyl, C₂-C₆ straight or branched alkenyl, C₁-C₆ straight or branched alkynyl, and —Si(C₁-C₄ alkyl)₃;
n1 is an integer independently selected in each instance from the group of 1, 2, 3, and 4;
n2 is an integer independently selected in each instance from the group of 1, 2, 3, 4, 5, 6, 7, 8, 9, and 10;
n3 is an integer independently selected in each instance from the group of 1, 2, 3, 4, 5, 6, 7, 8, 9, and 10;
n4 is an integer independently selected in each instance from the group of 1, 2, 3, 4, 5, 6, 7, 8, 9, and 10; and
n5 is an integer independently selected in each instance from the group of 1, 2, 3, 4, 5, 6, 7, 8, 9, and 10;
with the proviso that the sum of n2+n4 is not greater than 10;
with the proviso that the sum of n3+n5 is not greater than 10;
with the proviso that the sum of n2+n5 is not greater than 10; and
with the proviso that the sum of n3+n4 is not greater than 10.

3. A compound of claim 2, wherein:
R₁ is selected from the group of methyl, ethyl, n-propyl, isopropyl, —(CH₂)ₙ₁—SO₃⁻, —(CH₂)ₙ₁—N⁺(CH₃)₃;
—CH₂—CH₂—O—CH₃;
—CH₂—CH₂—O—CH₂—CH₃,
—CH₂—CH₂—O—[CH₂—CH₂—O]ₙ₂—CH₃;
—CH₂—CH₂—O—[CH₂—CH₂—O]ₙ₂—CH₂—CH₃;
—CH₂—CH₂—CH₂—O—CH₃;
—CH₂—CH₂—CH₂—O—CH₂—CH₃;
—CH₂—CH₂—CH₂—O—[CH₂—CH₂—CH₂—O]ₙ₃—CH₃; and
—CH₂—CH₂—CH₂—O—[CH₂—CH₂—CH₂—O]ₙ₃—CH₂—CH₃;
R₂ is selected from the group of —(CH₂)ₙ₁—SO₃⁻, —(CH₂)ₙ₁—N⁺(CH₃)₃;
—CH₂—CH₂—O—CH₃;
—CH₂—CH₂—O—CH₂—CH₃,
—CH₂—CH₂—O—[CH₂—CH₂—O]ₙ₄—CH₃;
—CH₂—CH₂—O—[CH₂—CH₂—O]ₙ₄—CH₂—CH₃;
—CH₂—CH₂—CH₂—O—CH₃;
—CH₂—CH₂—CH₂—O—CH₂—CH₃;
CH₂—CH₂—CH₂—O—[CH₂—CH₂—CH₂—O]ₙ₅—CH₃; and
—CH₂—CH₂—CH₂—O—[CH₂—CH₂—CH₂—O]ₙ₅—CH₂—CH₃;
n1 is an integer independently selected in each instance from the group of 1, 2, 3, and 4;
n2 is an integer independently selected in each instance from the group of 1, 2, 3, 4, 5, 6, 7, 8, 9, and 10;
n3 is an integer independently selected in each instance from the group of 1, 2, 3, 4, 5, 6, 7, 8, 9, and 10;
n4 is an integer independently selected in each instance from the group of 1, 2, 3, 4, 5, 6, 7, 8, 9, and 10; and
n5 is an integer independently selected in each instance from the group of 1, 2, 3, 4, 5, 6, 7, 8, 9, and 10;
with the proviso that the sum of n2+n4 is not greater than 10;
with the proviso that the sum of n3+n5 is not greater than 10;
with the proviso that the sum of n2+n5 is not greater than 10; and
with the proviso that the sum of n3+n4 is not greater than 10.

4. The compound of claim 2, wherein:
R₁ is selected from the group of methyl, ethyl, n-propyl, isopropyl, —(CH₂)ₙ₁—SO₃⁻, —(CH₂)ₙ₁—N⁺(CH₃)₃;
—CH₂—CH₂—O—CH₃;
—CH₂—CH₂—O—CH₂—CH₃,
—CH₂—CH₂—O—[CH₂—CH₂—O]ₙ₂—CH₃;
—CH₂CH₂—O—[CH₂—CH₂—O]ₙ₂—CH₂—CH₃;
—CH₂—CH₂—CH₂—O—CH₃;
—CH₂—CH₂—CH₂—O—CH₂—CH₃;
—CH₂—CH₂—CH₂—O—[CH₂—CH₂—CH₂—O]ₙ₃—CH₃; and
—CH₂—CH₂—CH₂—O—[CH₂—CH₂—CH₂—O]ₙ₃—CH₂—CH₃;
R₂ is selected from the group of —(CH₂)ₙ₁—SO₃⁻, —(CH₂)ₙ₁—N⁺(CH₃)₃;
—CH₂—CH₂—O—CH₃;
—CH₂—CH₂—O—CH₂—CH₃,
—CH₂—CH₂—O—[CH₂—CH₂—O]ₙ₄—CH₃;
—CH₂CH₂—O—[CH₂—CH₂—O]ₙ₄—CH₂—CH₃;
—CH₂—CH₂—CH₂—O—CH₃;
—CH₂—CH₂—CH₂—O—CH₂—CH₃;
—CH₂—CH₂—CH₂—O—[CH₂—CH₂—CH₂—O]ₙ₅—CH₃; and
—CH₂—CH₂—CH₂—O—[CH₂—CH₂—CH₂—O]ₙ₅—CH₂—CH₃;
n1 is an integer independently selected in each instance from the group of 1, 2, 3, and 4;
n2 is an integer independently selected in each instance from the group of 1, 2, 3, 4, 5, 6, 7, and 8;
n3 is an integer independently selected in each instance from the group of 1, 2, 3, 4, 5, 6, 7, and 8;
n4 is an integer independently selected in each instance from the group of 1, 2, 3, 4, 5, 6, 7, and 8; and
n5 is an integer independently selected in each instance from the group of 1, 2, 3, 4, 5, 6, 7, and 8;
with the proviso that the sum of n2+n4 is not greater than 10;
with the proviso that the sum of n3+n5 is not greater than 10;
with the proviso that the sum of n2+n5 is not greater than 10; and
with the proviso that the sum of n3+n4 is not greater than 10.

5. The compound of claim 1, having Formula (III) 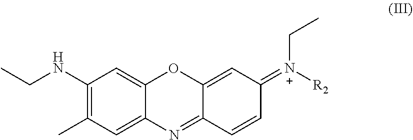
wherein:
R₂ is selected from the group of —(CH₂)ₙ₁—SO₃⁻, —(CH₂)ₙ₁—N⁺(CH₃)₃;
CH₂—CH₂—O—CH₃;
CH₂—CH₂—O—CH₂—CH₃,
—CH₂—CH₂—O—[CH₂—CH₂—O]ₙ₄—CH₃;
CH₂CH₂—O—[CH₂—CH₂—O]ₙ₄—CH₂—CH₃;
CH₂—CH₂—CH₂—O—CH₃;
CH₂—CH₂—CH₂—O—CH₂—CH₃;
CH₂—CH₂—CH₂—O—[CH₂—CH₂—CH₂—O]ₙ₅—CH₃; and
CH₂—CH₂—CH₂—O—[CH₂—CH₂—CH₂—O]ₙ₅—CH₂—CH₃;
n1 is an integer independently selected in each instance from the group of 1, 2, 3, and 4;
n4 is an integer independently selected in each instance from the group of 1, 2, 3, 4, 5, 6, 7, and 8; and
n5 is an integer independently selected in each instance from the group of 1, 2, 3, 4, 5, 6, 7, and 8.

6. The compound of claim 5, wherein:
R₂ is selected from the group of —(CH₂)ₙ₁—SO₃⁻, —(CH₂)ₙ₁—N⁺(CH₃)₃;
CH₂—CH₂—O—CH₃;
CH₂—CH₂—O—CH₂—CH₃,
—CH₂—CH₂—O—[CH₂—CH₂—O]ₙ₄—CH₃;
CH₂CH₂—O—[CH₂—CH₂—O]ₙ₄—CH₂—CH₃;
CH₂—CH₂—CH₂—O—CH₃;
CH₂—CH₂—CH₂—O—CH₂—CH₃;
CH₂—CH₂—CH₂—O—[CH₂—CH₂—CH₂—O]ₙ₅—CH₃; and
CH₂—CH₂—CH₂—O—[CH₂—CH₂—CH₂—O]ₙ₅—CH₂—CH₃;
n1 is an integer independently selected in each instance from the group of 1, 2, 3, and 4;
n4 is an integer independently selected in each instance from the group of 1, 2, 3, 4, 5, and 6; and
n5 is an integer independently selected in each instance from the group of 1, 2, 3, 4, 5, and 6.

7. The compound of claim 5, wherein:
R₂ is selected from the group of —(CH₂)ₙ₁—SO₃⁻, —(CH₂)ₙ₁—N⁺(CH₃)₃;
CH₂—CH₂—O—CH₃;
CH₂—CH₂—O—CH₂—CH₃,
—CH₂—CH₂—O—[CH₂—CH₂—O]ₙ₄—CH₃;
CH₂CH₂—O—[CH₂—CH₂—O]ₙ₄—CH₂—CH₃;
CH₂—CH₂—CH₂—O—CH₃;
CH₂—CH₂—CH₂—O—CH₂—CH₃;
CH₂—CH₂—CH₂—O—[CH₂—CH₂—CH₂—O]ₙ₅—CH₃; and
CH₂—CH₂—CH₂—O—[CH₂—CH₂—CH₂—O]ₙ₅—CH₂—CH₃;
n1 is an integer independently selected in each instance from the group of 1, 2, 3, and 4;
n4 is an integer independently selected in each instance from the group of 1, 2, 3, and 4; and
n5 is an integer independently selected in each instance from the group of 1, 2, 3, and 4.

8. The compound of claim 1, having Formula (VI): 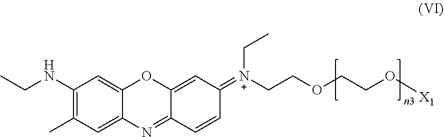
wherein X₁ is selected from the group of C₁-C₆ straight or branched alkyl, C₂-C₆ straight or branched alkenyl, C₁-C₆ straight or branched alkynyl, and —Si(C₁-C₄ alkyl)₃; and
n3 is an integer selected from the group of 1, 2, 3, 4, 5, 6, 7, 8, 9, and 10.

9. The compound of claim 1, having Formula (VII): 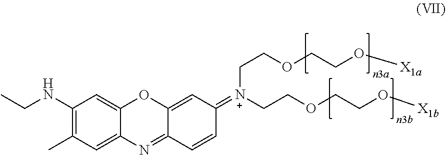
wherein X₁ₐ and X₁b are selected independently from the group of C₁-C₆ straight or branched alkyl, C₂-C₆ straight or branched alkenyl, C₁-C₆ straight or branched alkynyl, and —Si(C₁-C₄ alkyl)₃; and
n3a and n3b are each an integer independently selected from the group of 1, 2, 3, 4, 5, 6, 7, 8, 9, and 10; with the proviso that the sum of n3a+n3b is not greater than 10.

10. The compound of claim 1, having Formula (IV): 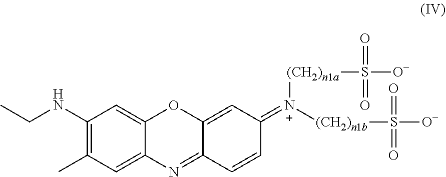
wherein n1a and n1b are each integers independently selected from the group of 1, 2, 3, and 4.

11. The compound of claim 1, having Formula (V): 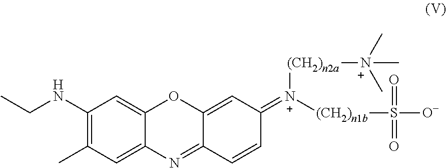
wherein n2a and n1b are each integers independently selected from the group of 1, 2, 3, and 4.

12. The compound of claim 1 selected from the group of:

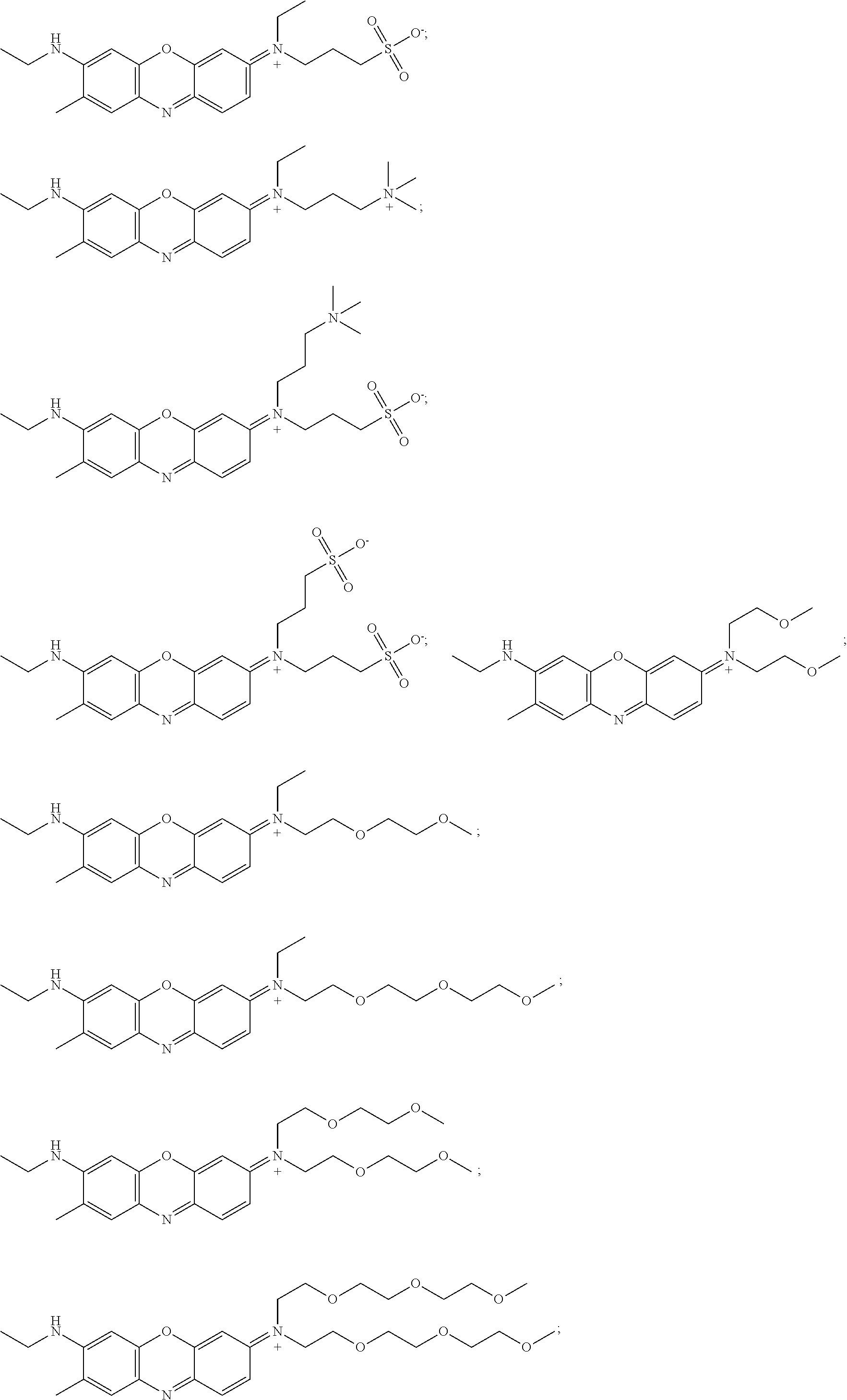

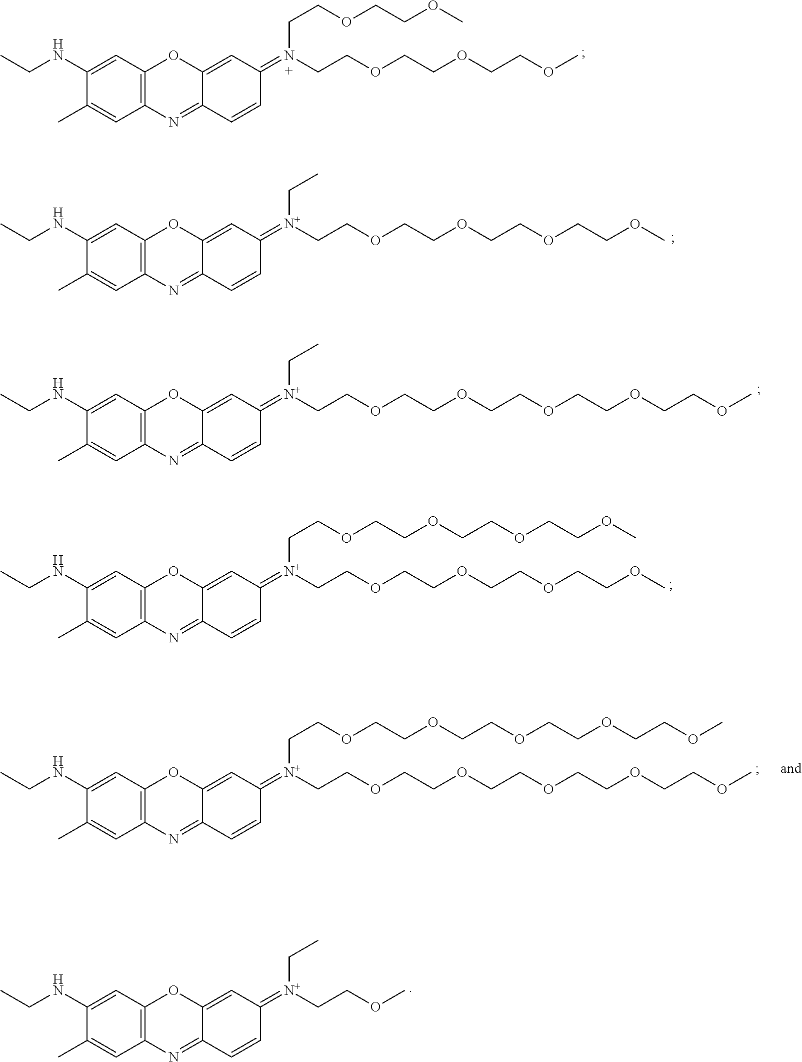

13. A method of detecting nerves intraoperatively in a subject undergoing surgery, the method comprising:
a) systemically administering an effective amount of a composition comprising a compound of claim 1 to the subject before or during surgery to form a stained tissue; and
b) imaging the stained tissue undergoing surgery in the subject, thereby detecting nerves intraoperatively in the subject undergoing surgery.

14. A composition comprising an effective amount of a compound of claim 1 and a pharmaceutically acceptable carrier or excipient.
